(19) United States
(12) Patent Application Publication
(10) Pub. No.: US 2022/0313789 A1
(43) Pub. Date: Oct. 6, 2022

(54) TARGETING MELANOCORTIN 3 RECEPTOR FOR TREATMENT/PREVENTION OF EATING, METABOLISM, AND/OR EMOTIONAL DISORDERS

(71) Applicant: The Regents of the University of Michigan, Ann Arbor (US)

(72) Inventors: Roger Cone, Ann Arbor, MI (US); Patrick Sweeney, Ann Arbor, MI (US)

(21) Appl. No.: 17/619,175

(22) PCT Filed: Jun. 19, 2020
(86) PCT No.: PCT/US2020/038757

§ 371 Date: Dec. 14, 2021

Related U.S. Application Data
(60) Provisional application No. 62/863,388, filed on Jun. 19, 2019.
(60) Provisional application No. 62/902,088, filed on Sep. 18, 2019.

(51) Int. Cl. A61K 38/34; A61K 38/26; A61K 31/485; A61K 45/06; A61P 25/24; A61P 3/04

(52) U.S. Cl. CPC A61K 38/34; A61K 38/26; A61K 31/485; A61K 45/06; A61P 25/24; A61P 3/04

(57) ABSTRACT

Provided herein are compositions and methods for targeting (e.g., inhibiting or enhancing the activity or expression N of) melanocortin 3 receptor (MC3R) gene, mRNA, and protein for the treatment and/or prevention of eating disorders (e.g., anorexia nervosa, cachexia, etc.), metabolic disorders (e.g., obesity, diabetes, non-alcoholic steatohepatitis, hypertension, etc.), and/or emotional/mental disorders (e.g., depression, anxiety, OCD, PTSD, etc.). In particular, provided herein are MC3R agonists that stimulate MC3R for the treatment/prevention of disorders such as anorexia nervosa and other eating and/or anxiety disorders, MC3R antagonists that inhibit MC3R for the treatment/prevention of obesity and/or other eating/metabolism disorders, and methods of use thereof.

### CROSS-REFERENCE TO RELATED APPLICATIONS

The present application claims the priority benefit of U.S. Provisional Patent Application 62/902,088, filed Sep. 18, 2019, and U.S. Provisional Patent Application 62/863,388, filed Jun. 19, 2019, each of which is incorporated by reference in its entirety.

### FIELD

Provided herein are compositions and methods for targeting (e.g., inhibiting or enhancing the activity or expression of) melanocortin 3 receptor (MC3R) gene, mRNA, and/or protein for the treatment and/or prevention of eating disorders (e.g., anorexia nervosa, cachexia, etc.), metabolic disorders (e.g., obesity, diabetes, non-alcoholic steatohepatitis, hypertension, etc.), and/or emotional/mental disorders (e.g., depression, anxiety, OCD, PTSD, etc.). In particular, provided herein are MC3R agonists that stimulate MC3R for the treatment/prevention of disorders such as anorexia nervosa and other eating and/or anxiety disorders, MC3R antagonists that inhibit MC3R for the treatment/prevention of obesity and/or other eating/metabolism disorders, and methods of use thereof.

### BACKGROUND

Disorders of negative energy balance, such as anorexia nervosa and disease cachexia, are characterized by decreased food intake, dangerously low BMI, and an increased risk of anxiety and depression. Despite the severe consequences of these disorders, few pharmacological strategies exist to stimulate feeding and reduce anxiety in these at-risk patient populations. A large body of research has established the critical role of hypothalamic

AgRP neural circuits in stimulating feeding and intense effort has focused on identifying pharmacological targets that suppress these circuits as potential therapeutics for obesity. However, the utility of pharmacological stimulation of these pathways in conditions of negative energy balance, such as anorexia nervosa or disease cachexia, has been much less studied.

### SUMMARY

Provided herein are compositions and methods for targeting (e.g., inhibiting or enhancing the activity or expression of) melanocortin 3 receptor (MC3R) gene, mRNA, and protein for the treatment and/or prevention of eating disorders (e.g., anorexia nervosa, cachexia, etc.), metabolic disorders (e.g., obesity, diabetes, non-alcoholic steatohepatitis, hypertension, etc.), and/or emotional/mental disorders (e.g., depression, anxiety, OCD, PTSD, etc.). In particular, provided herein are MC3R agonists that stimulate MC3R for the treatment/prevention of disorders such as anorexia nervosa and other eating and/or anxiety disorders, MC3R antagonists that inhibit MC3R for the treatment/prevention of obesity and/or other eating/metabolism disorders, and methods of use thereof.

In some embodiments, provided herein are methods of treating an eating disorder comprising administering a melanocortin 3 receptor (MC3R) agonist to a subject suffering from the eating disorder. In some embodiments, the eating disorder is characterized by under eating. In some embodiments, the eating disorder is characterized by one or more emotional/mental symptoms. In some embodiments, the eating disorder is characterized by anxiety and/or depression. In some embodiments, the eating disorder is anorexia nervosa. In some embodiments, the eating disorder is cachexia. In some embodiments, the eating disorder is stress-induced anorexia. In some embodiments, the MC3R agonist is selective for MC3R over melanocortin 4 receptor (MC4R). In some embodiments, the MC3R agonist is a peptide. In some embodiments, the peptide comprises an amino acid sequence of SEQ ID NOS: 1-15. In some embodiments, the peptide comprises an amino acid sequence of SEQ ID NO: 12, wherein Xaa¹ and Xaa⁴ are selected from Table 3. In some embodiments, the MC3R agonist is a small molecule. In some embodiments, the MC3R agonist is a natural product. In some embodiments, the administration is repeated on a recurring basis for a period of at least 1 week (e.g., 1 week, 2 weeks, 1 month, 2 months, 4 months, 6 months, 9 months, 1 year, 2 years, 3, years, 4 years, or more). In some embodiments, the administration is repeated on a daily basis. In some embodiments, the administration is repeated on a twice-daily basis. In some embodiments, the administration is repeated on alternate days. In some embodiments, the administration is repeated on a weekly basis. In some embodiments, the administration is repeated on a recurring basis for a period of at least 1 month (e.g., 1 month, 2 months, 4 months, 6 months, 9 months, 1 year, 2 years, 3, years, 4 years, or more). In some embodiments, the administration is repeated on a recurring basis for a period of at least 1 year. In some embodiments, the MC3R agonist is co-administered with nutritional therapy, psychotherapy, nasogastric feeding, antidepressant agents, and/or antipsychotic agents.

In some embodiments, provided herein are methods of treating an emotional/mental disorder comprising administering a melanocortin 3 receptor (MC3R) agonist to a subject suffering from the emotional/mental disorder. In some embodiments, the eating disorder is characterized by anxiety and/or depression. In some embodiments, the MC3R agonist is selective for MC3R over melanocortin 4 receptor (MC4R). In some embodiments, the MC3R agonist is a peptide. In some embodiments, the peptide comprises an amino acid sequence of SEQ ID NOS: 1-15. In some embodiments, the peptide comprises an amino acid sequence of SEQ ID NO: 12, wherein Xaa¹ and Xaa⁴ are selected from Table 3. In some embodiments, the MC3R agonist is a small molecule. In some embodiments, the administration is repeated on a recurring basis for a period of at least 1 week (e.g., 1 week, 2 weeks, 1 month, 2 months, 4 months, 6 months, 9 months, 1 year, 2 years, 3, years, 4 years, or more). In some embodiments, the administration is repeated on a daily basis. In some embodiments, the administration is repeated on a twice-daily basis. In some embodiments, the administration is repeated on a weekly basis. In some embodiments, the administration is repeated on a recurring basis for a period of at least 1 month (e.g., 1 month, 2 months, 4 months, 6 months, 9 months, 1 year, 2 years, 3, years, 4 years, or more). In some embodiments, the administration is repeated on a recurring basis for a period of at least 1 year. In some embodiments, the MC3R agonist is co-administered with psychotherapy (e.g., cognitive behavioral therapy, family therapy, etc.), antianxiety agents, mood stabilizers, stimulants, antidepressant agents, and/or antipsychotic agents.

In some embodiments, provided herein are methods of treating an eating disorder comprising administering a melanocortin 3 receptor (MC3R) antagonist to a subject suffering from the eating disorder. In some embodiments, the eating disorder is characterized by over eating. In some embodiments, the eating disorder is characterized by obesity. In some embodiments, provided herein are methods of treating obesity in a subject comprising administering a melanocortin 3 receptor (MC3R) antagonist to a subject suffering from obesity. In some embodiments, the administration is repeated on a recurring basis for a period of at least 1 week (e.g., 1 week, 2 weeks, 1 month, 2 months, 4 months, 6 months, 9 months, 1 year, 2 years, 3, years, 4 years, or more). In some embodiments, the administration is repeated on a daily basis. In some embodiments, the administration is repeated on a twice-daily basis. In some embodiments, the administration is repeated on a weekly basis. In some embodiments, the administration is repeated on a recurring basis for a period of at least 1 month (e.g., 1 month, 2 months, 4 months, 6 months, 9 months, 1 year, 2 years, 3, years, 4 years, or more). In some embodiments, the administration is repeated on a recurring basis for a period of at least 1 year. In some embodiments, the MC3R antagonist is co-administered with an appetite suppressant. In some embodiments, the MC3R antagonist is co-administered with an anti-anxiety medication. In some embodiments, the MC3R antagonist is co-administered with a Glp1 agonist, such as liraglutide. In some embodiments, MC3R activity is blocked or reduced using antisense mRNA or oligonucleotides.

In some embodiments, provided herein are pharmaceutical compositions comprising an MC3R antagonist and a weight-loss drug. In some embodiments, the MC3R antagonist and a weight-loss drug are separately formulated. In some embodiments, the MC3R antagonist and a weight-loss drug are in a single formulation. In some embodiments, the weight loss drug is a glucagon-like peptide 1 (GLP-1) receptor agonist. In some embodiments, the GLP-1 receptor agonist is selected from aglutide, dulaglutide, exenatide, exenatide extended release, semaglutide, and lixisenatide. In some embodiments, the weight loss drug is Contrave (Naltrexone Hydrochloride and Bupropion Hydrochloride), Qysmia (Phentermine and Topiramate), or Belviq (lorcaserin hydrochloride). In some embodiments, provided herein are A methods of treating obesity and/or inducing weight loss comprising administering any of the aforementioned pharmaceutical compositions.

In some embodiments, provided herein are methods of treating obesity and/or inducing weight loss comprising co-administering an MC3R antagonist and a weight-loss drug. In some embodiments, the weight loss drug is a glucagon-like peptide 1 (GLP-1) receptor agonist. In some embodiments, the GLP-1 receptor agonist is selected from aglutide, dulaglutide, exenatide, exenatide extended release, semaglutide, and lixisenatide. In some embodiments, the weight loss drug is Contrave (Naltrexone Hydrochloride and Bupropion Hydrochloride), Qysmia (Phentermine and Topiramate), or Belviq (lorcaserin hydrochloride).

### BRIEF DESCRIPTION OF THE DRAWINGS

FIG. 1. MC3R is widely expressed in a variety of arcuate neurons. Enriched expression is observed in orexigenic AgRP neurons.

FIG. 2. Potent Bidirectional Regulation of Feeding by DREADD Activation of MC3R ARC neurons. (A) Representative images showing hM3Dq-mCherry (red), and cfos (green), in the arcuate nucleus of a mouse transduced with hM3Dq in arcuate MC3R expressing neurons following CNO or saline administration. Panels on right are an enlargement of the boxed sections. (B) Quantification of cfos expression in the arcuate nucleus following i.p. injections of saline or CNO (unpaired Student's t-test). CNO injections increased cfos expression in the arcuate nucleus. (C) Acute feeding assays following DREADD mediated activation of arcuate MC3R neurons. Activation of MC3R neurons increased food intake at all times tested (2-way ANOVA with Bonferroni post-hoc test, n=10 mice). (D) Change in body weight following a single injection of CNO or saline. Body weight increased on the day following CNO injection, remaining elevated five days following CNO injection (2 way ANOVA with Bonferroni post-hoc test, n=10 mice). (E) Daily food intake in MC3R-Cre mice targeted with the inhibitory DREADD hM4Di in arcuate MC3R expressing neurons. Twice daily injections of CNO reduced daily food intake in hM4Di transfected mice, relative to WT control animals (unpaired Student's t-test, n=6 mice for WT group and n=5 mice for hM4Di group). (F) Change in body weight following long term CNO injections in hM4Di transfected and WT mice. CNO injections reduced BW in hM4Di transfected mice, relative to WT control mice (2-way ANOVA, n=6 mice for WT group and n=5 mice for hM4Di group). CNO administered at 0.1 mg/kg in 200 ul saline for all panels. All mice between 10-14 weeks old.

FIG. 3. Arcuate MC3R neurons can bidirectionally regulate anxiety. A: Entries to the open arms and time in the open arms in mice targeted with hM3Dq in ARC-MC3R neurons during elevated plus maze testing (EPM). DREADD mediated activation of ARC-MC3R neurons increased entries to the open arms (left) and time in the open arms (right). B: Entries to the center and time in the center during open field testing (OFT). DREADD activation of ARC-MC3R neurons increased entries to the center (left) and time in the center (right). C: heat plot showing average time in each area of the open field during open field testing. D: Time in the open arms (left) and distance traveled in the open arms (right) during elevated plus maze testing in mice targeted with hM4Di in ARC-MC3R neurons. Chemogenetic inhibition of ARC-MC3R neurons decreased time in the open arms and decreased distance traveled in the open arms. E: Time in the center and distance traveled in the center during open field testing. Chemogenetic inhibition of ARC-MC3R neurons reduced time in the center and distance traveled in the center. F: Heat plot showing average amount of time spent in each area of the open field for WT and hM4Di transduced mice. Data represents mean+/−s.e.m. CNO (1mg/kg, i.p.) was administered ten minutes prior to testing in all mice. Data analyzed with Student's unpaired t-test. *p<0.05, **p<0.01. n=11 mice for WT and n=11 mice for hM3Dq groups (panels A-C). n=14 mice for WT and n=15 mice for hM4Di (panels D-F).

FIG. 4. Lack of MC3R results in elevated anxiety. A: Total distance traveled (left), time in open arms (middle), and entries to the open arms (right) in WT and MC3R KO mice during elevated plus maze (EPM) testing. MC3R KO mice travel less distance while spending less time in the open arms and entering the open arms less frequently. B: Total distance traveled (left panel), time in center (middle panel), and entries to the center (right panel) during open field testing (OFT) in WT and MC3R KO mice. MC3R KO mice spent less time in the center and entered the center less frequently. No significant difference was observed between WT and MC3R KO mice in total distance traveled (B). C: Heat plot showing average amount of time spent in the open field arena for WT and MC3R KO mice. Data is represented as mean+/−s.e.m. All panels analyzed with Student's unpaired t-test. n=22 mice for WT group and n=20 mice for MC3R KO group.

FIG. 5. Lack of MC3R exacerbates stress-induced anorexia. A: Body weight of WT and MC3R KO mice prior to social isolation induced anorexia experiments. B: Food intake post single housing in WT and MC3R KO mice. MC3R KO mice consumed less food than their WT counterparts immediately following single housing. C: Change in BW in WT and

MC3R KO mice 24 hours after single housing. MC3R KO mice lost significantly more weight than WT mice in response to social isolation stress. D: Daily food intake prior and in the days following social isolation. While daily food intake was not different between WT and MC3R KO mice prior to social isolation stress, MC3R KO mice ate less than WT mice following social isolation stress. E: Change in BW 24 hours post restraint stress. MC3R KO mice lose significantly more weight in response to acute restraint stress than WT mice. F: Food intake 2 hours following restraint stress in WT and MC3R KO mice. MC3R KO mice eat less than WT mice following acute restraint stress. G and H: Change in BW during chronic daily restraint stress in WT and MC3R KO males (G) and females (H). MC3R KO males lose more weight than WT mice during daily restraint stress (G). No significant difference in weight change in response to daily restraint stress was detected between WT and MC3R KO female mice (H). Data represented as mean+/−s.e.m. n=15 mice for WT group in panels A-D and n=16 mice for MC3R KO group in panels A-D. n=15 mice for no restraint WT group, n=16 mice for no restraint MC3R KO group, n=15 mice for restraint WT group, and n=19 mice for restraint MC3R KO group for panel E. n=8 cages for MC3R KO and n=4 cages for WT groups in panel F. n=8 mice for WT group and n=9 mice for MC3R KO group in panel G. n=7 WT mice and n=11 MC3R KO mice for panel H. Panels A, C, and F, analyzed with Student's unpaired t-test. Panels B, D, E, G, and H analyzed with two-way ANOVA with Tukey's post hoc test.

FIG. 6. Melanocortin 3 receptor agonists increase feeding and body weight. A and B: Acute food intake in response to peripheral administration of the MC3R agonists PG990 (A, 10 mg/kg; i.p., n=12 mice) and FMMC-5 (B, 10 mg/kg, i.p., n=12 mice). Both MC3R agonists acutely stimulated food intake relative to vehicle control injections. C: Feeding in response to central administration of the MC3R agonist PG992 (i.c.v., 5 ug in 500 nl DMSO) or vehicle (500 nl DMSO). PG992 administration acutely stimulated feeding at all measured time-points post injection (n=8 mice). D and E: Food intake at 4 and 24 hours following central administration of PG992. PG992 administration increased food intake at both 4 hours and 24 hours post injection (n=27 mice). F and G: Daily food intake (F) and change in body weight (G) during once daily i.c.v. injections (5 ug) of the MC3R agonist PG992. Administration of PG992 increased daily food intake (F, n=8 mice) and body weight (G, n=8 mice). Body weight was increased by nearly 8 percent following three days of PG992 treatment (G). H: Food intake in response to chemogenetic activation of AgRP neurons vs. PG992 mediated stimulation of MC3R. Chemogenetic activation of AgRP neurons produced a more robust acute hyperphagic response than activation of MC3R (H; n=8 mice per group). I: 24-hour food intake following chemogenetic activation of AgRP neurons or central administration of PG992. PG992 increased 24-hour food intake to a greater extent than chemogenetic activation of AgRP neurons. Data represents mean+/−s.e.m. Experiments performed in ad libitum fed mice during the rodent light cycle. Panels D and E analyzed by paired Student's t-test. All other panels analyzed by 2-way ANOVA with Tukey's post hoc test.

FIG. 7. MC3R-specific agonists stimulate food intake via AgRP neurons. A: Representative image showing expression of hM4Di in arcuate AgRP neurons. B-D: 4-hour food intake (B), 8-hour food intake, (C) following vehicle or PG992 injections (5 ug), and (D), and 24-hour food intake. Either saline or CNO (1 mg/kg, i.p.) was administered fifteen minutes prior to i.c.v. injections. PG992 increased food intake following both saline and CNO injections at all time-points, although PG992 induced stimulation of food intake was significantly reduced in the presence of AgRP neuron inhibition. Data represented as mean+/−s.e.m. Data analyzed with 2-way ANOVA with Tukey's post hoc test. n=9 mice for panels B-D.

FIG. 8. Melanocortin 3 receptor activation reduces anxiety. A: Central administration of the MC3R agonist PG992 (5 ug, i.c.v.) reduced anxiety in the open field test (OFT). Acute PG992 administration reduced latency to enter the center of the arena, and increased distance traveled in the center and entries in the center. Total distance traveled was also increased following PG992 treatment (n=17 mice for vehicle group and n=15 mice for PG992 group). B: Acute administration of PG992 reduced anxiety in the elevated plus maze (EPM). Mice treated with PG992 (5 ug, i.c.v.) entered the open arms faster, traveled more distance in the open arms, and entered the open arms more frequently. Consistent with the open field test, treatment with PG992 also increased total distance traveled (n=20 mice for vehicle group and n=20 mice for PG992 group). C: Stimulation of MC3R reduced anxiety and increased food intake in the novelty suppressed feeding test (NSF). Acute stimulation of MC3R reduced latency to eat in the NSF test and increased total time eating. In contrast to OFT and EPM results, treatment with PG992 did not affect distance traveled in the NSF test. Data represents mean+/−s.e.m. Experiments performed in ad libitum fed mice during the rodent light cycle. All panels analyzed by unpaired Student's t-test.

FIG. 9. CNO administration does not affect food intake in WT mice. 24-hour food intake following i.p. injections of saline or CNO. No difference in food intake was detected between the two treatment conditions. Data represented as mean+/−s.em. Data analyzed with Student's paired t-test. n=15 mice

FIG. 10. Chemogenetic activation of ARC-MC3R cells decrease latency to eat and suppresses anxiety in the novelty suppressed feeding assay. Latency to eat (left panel) and time in the center (right panel) during ten minutes of exploration in a novelty suppressed feeding assay. Stimulation of ARC-MC3R neurons decreased latency to eat and increased time spent in the center of the open field. Data represented as mean+/−s.e.m. Data analyzed with Student's unpaired t-test. **p<0.01. n=11 mice for hM3Dq group and n=8 mice for WT group.

FIG. 11. DREADD-mediated activation or inhibition of ARC-MC3R neurons does not affect locomotion in the elevated plus maze and open field test. A: Total distance traveled following chemogenetic activation (top panel) or inhibition (bottom panel) in the elevated plus maze test. No significant differences were detected in locomotion following either chemogenetic activation (top panel) or inhibition (bottom panel). B: Total distance traveled in the open field test during chemogenetic activation (top panel) or inhibition (bottom panel) or ARC-MC3R neurons. No significant differences were detected in distance traveled in any of the groups. Data represents mean+/−s.e.m. Data analyzed with Student's unpaired t-test. n=16 mice for hM4Di group and n=13 mice for WT group (bottom panels). n=11 mice in WT group and n=11 mice in hM3Dq group (top panels).

FIG. 12. Enhanced social isolation induced anorexia and anxiety in MC3R TB/TB mice. A: Body weight of WT and MC3R TB/TB mice prior to social isolation. No significant difference in BW was observed between the two groups. B: 24-hour food intake during social isolation in WT and MC3R TB/TB mice. MC3R TB/TB mice consume less food during social isolation than WT mice. C: Total distance traveled during elevated plus maze testing in WT and MC3R TB/TB mice. No difference in total distance traveled was detected between the two groups. D: Entries to the open arms during elevated plus maze testing. A trend towards reduced entries into the open arms was observed in the MC3R TB/TB mice. E: Latency to enter the open arms during open field testing. MC3R TB/TB mice entered the open arms later than WT mice. F: Time in the closed arms during EPM testing. MC3R TB/TB mice spent more time than WT mice in the closed arms. Data represented as mean+/−s.e.m. Data analyzed with Student's unpaired t-test. n=13 mice for WT group and n=16 mice for MC3R TB/TB group in panels A and B. n=13 mice for WT group and n=12 mice for MC3R TB/TB group in panels C-F.

FIG. 13. MC3R agonist PG992 does not affect feeding in MC3R KO mice. A: Acute food intake in MC3R KO mice following i.c.v. administration of vehicle (DMSO, 500 nl) or PG992 (5 ug in 500 nl DMSO). No significant difference in food intake was detected between vehicle and PG992 treatment in MC3R KO mice. B: 24-hour food intake following i.c.v. vehicle (DMSO, 500 nl) or PG992 injection (5 ug in 500 nl DMSO). No significant difference in 24-hour food intake was detected between vehicle and PG992 groups. C: Representative image showing hM3Dq-mCherry expression in arcuate AgRP neurons in an AgRP-Cre mouse. Data shown as mean+/−s.e.m. Panels A and B analyzed with Student's paired t-test. n=9 mice for panels A and B.

FIG. 14. MC3R and behavioral control. MC3R is expressed pre-synaptically on AgRP and POMC neuronal projections to brain regions controlling reproduction (blue), feeding and metabolism, (purple), and behavioral control centers (green). MC3R is also expressed postsynaptically within some of these brain regions. AVPV, Anteroventral periventricular nucleus; PMv, ventral premammillary nucleus; ARC, arcuate nucleus; PVH, paraventricular nucleus; GnRH, gonadotropin releasing hormone; PVT, paraventricular nucleus of the thalamus; PB1 lateral parabrachial nucleus; VTA, ventral tegmental area; ER, estrogen receptor; AR, androgen receptor; LRb, leptin receptor.

FIG. 15. MC3R at the intersection of feeding behavior and reproduction. MC3R is expressed pre-synaptically on AgRP and POMC neurons driving feeding behavior (purple), that also project to brain regions controlling reproduction (blue), expressing MC3R as well.

FIG. 16. Sexual dimorphism of MC3R Expression in humans and mice. (A) Transcriptomics data from the GTEX consortium shows a 2-3× higher mean expression of MC3R mRNA in male (blue) vs female (red) hypothalamus. Y axis is a log scale. (B-G) MC3R-GFP positive cells in hypothalamic arcuate nucleus of male (B) and female (C) MC3R-GFP mice. 3× more GFP IHC-positive cells are observed per section when averaged across 6 independent animals (D, 8-10 week old mice, unpaired Student's t-test). In situ hybridization to detect native MC3R transcript in male (E) and female (F) hypothalamus. Hybridization density is 2× greater in male vs female mice (G; n=4-5 mice each, 8-10 weeks old, unpaired Student's t-test), following normalization to background. *p<0.05, **p<0.01

FIG. 17. Deletion of the MC3R produces sexually dimorphic anxiety and hypophagia in novelty suppressed feeding test. (A and B) Latency to enter the food zone (A) and latency to consume food (B) during NSF tests. MC3R KO female mice took significantly longer to enter the food zone (A) than all other groups tested and failed to consume food during testing. (C and D) Time in the center of the open field (C) and total distance traveled (D) during testing. MC3R KO females spent less time in the center (C) and traveled less (D) than all other groups. Data analyzed with 2-way ANOVA. n=11 mice for male WT, n=8 mice for female WT, n=11 mice for male MC3R KO, n=8 mice for female KO. All mice 10-14 weeks old.

FIG. 18. Primary HTS agonist assay validation. (A): Concentration response curve for α-MSH using the primary HTS assay cell line. EC₅₀ range for αMSH is 4.75 to 5nM (95% CI). (B): Positive and negative assay control validation run with threshold bands and assay window indicated. (C): Response histograms for positive and negative controls from the data in panel “B”. Typical Z′ factors are above 0.7. (right panel) (D) Pilot screen assay heat map. Individual assay plates are represented by each row, while the well position per plate is mapped to each column. (E): Scatterplot of corrected and normalized data from panel (D). Hit assignment threshold was set to 3SD from negative control mean response. With this threshold, six “hits” were identified, with most potent compound indicated. (F): Raw kinetic response for CCG-106076 and closes neighboring negative and positive control wells.

FIG. 19A-B. Natural products dominate hits in an HTS for MC3R antagonists. (A): Top MC3R specific hit compounds are highlighted (B): List of top hit compounds with corresponding chemical structures, activity and molecular weight. Top hits identified were all natural products.

FIG. 20. MC3R KO mice show hypersensitivity to the weight loss drug liraglutide. Left panel: Cumulative food intake following vehicle or liraglutide administration to MC3R TB/TB mice. Saline or liraglutide (0.2 mg/kg, i.p.) was administered once daily from days 1 to five. Liraglutide significantly reduced food intake during treatment. Food intake remained reduced in MC3R TB/TB mice for multiple days following cessation of drug treatment. Right panel: Cumulative food intake following liraglutide (0.2 mg/kg, i.p.) or saline administration to WT mice. Food intake was reduced during liraglutide treatment. However cumulative food intake returned to vehicle control levels within one day following cessation of liraglutide treatment. Data analyzed with two-way ANOVA with Tukey's posthoc test. n=7 mice for vehicle treatment and n=6 mice for liraglutide treatment in left panel. n=7 mice for both vehicle and liraglutide treatment groups in right panel.

FIG. 21. MC3R KO mice are hypersensitive to the weight loss effects of liraglutide. Change in body weight following once daily liraglutide administration to WT and MC3R

TB/TB mice. MC3R KO mice lost more weight during liraglutide administration and continued to maintain a lower body weight than WT mice following cessation of liraglutide treatment. Data analyzed with two-way ANOVA with tukey's post hoc test. n=6 mice for WT group and 7 mice for MC3R TB/TB group.

FIG. 22. Naturally occurring melanocortin peptides.

FIG. 23. Primary HTS antagonist assay. (A): Concentration response curve for α-MSH using the primary HTS assay cell line. EC₅₀ range for αMSH is 1.75 to 1.85nM (95% CI). (B): Positive and negative assay with kinetics of antagonist and agonist addition indicated. (C): Scatterplot of corrected and normalized data. Typical Z′ factors are above 0.5. (D): Histogram of data from panel C.

FIG. 24. Administration of the MC3R antagonist, Compound 11, inhibits food intake and reduces body weight. Vehicle or 5 ug of compound 11 was administered intracerebroventricularly in mice. N=10 mice per treatment. Compound 11 reduced food intake at every time point (left panel; 1, 2, 4, 6 hr), and reduced body weight at a time point 24hr after treatment (right). Data analyzed by paired Student's t-test.

FIG. 25. PVN MC4R neurons are inhibited in response to MC3R agonist PG990. (left panels): Heat map displaying relative fluorescence, determined by endomicroscopy (Inscopix) of individual PVN MC4R neurons for five minutes of baseline recording, followed by five minutes of recording in response to a saline injection. No significant difference was detected following saline injection (quantified in bottom panel). (right panels): Heat map showing fluorescence activity for five minutes of baseline activity before and after (vertical white line) ip administration of the MC3R agonist PG990 (10 mg/kg). Fluorescence activity was suppressed following PG990 administration (quantified in bottom panel). N=18 cells for panel A and n=13 cells for panel B. Data analyzed by paired Student's t-test.

FIG. 26. Pharmacological antagonism of MC3R enhances anorexic effects of liraglutide. Change in food intake relative to vehicle treatment following administration of liraglutide (s.c., 0.2 mg/kg), the MC3R antagonist compound 11 (C11, 5 ug, i.c.v.), or co-administration of liraglutide and C11. Both liraglutide and C11 reduced food intake relative to vehicle treatment. The anorexic effect of liraglutide was significantly enhanced with co- administration of the MC3R antagonist compound 11. *p<0.05. Data analyzed with repeated measures 2-way ANOVA. n=19 mice for each group.

FIG. 27. Pharmacological antagonism of MC3R enhances weight loss properties of liraglutide. 24-hour change in body weight following treatment with liraglutide (s.c., 0.2 mg/kg), the MC3R antagonist compound 11 (C11, 5 ug, i.c.v.), or co-administration of liraglutide and C11. Both liraglutide and C11 reduced body weight, while co-administration of C11 and liraglutide produced more significant weight loss than either compound alone. *p<0.05, Data analyzed with 1-way ANOVA. n=19 mice for each group.

FIG. 28A-J. MC3R specific compounds bi-directionally regulate feeding. A: Cumulative food intake in WT mice following administration of vehicle (aCSF) or the MC3R agonist compound 18 (i.c.v., 5 ug). Food intake is increased at 2 hours, 4 hours, and 6 hours following administration of C18. B: 24-hour food intake following administration of vehicle (aCSF) or C18 to WT mice. 24-hour food intake is increased in WT mice following administration of C18. C: Change in BW following injection of vehicle or C18 (5 ug). D and E: Cumulative food intake (D) and 24-hour food intake (E) following C18 or vehicle administration in MC3R KO mice. No difference in food intake was detected following C18 administration in MC3R KO mice. F: Cumulative food intake following administration of the MC3R antagonist C11 or vehicle. C11 reduced food intake in WT mice at 2 hours, 4 hours, and 6 hours post injection. G: 24-hour food intake in WT mice following administration of C11. H: Change in BW following injection of vehicle or C11 (5 ug). I and J: Food intake following i.c.v. injection of vehicle (DMSO, 500 nl) or C11 (5 ug, 500 nl) in MC3R KO mice. No difference in food intake was detected between vehicle and C11 treatment groups in MC3R KO mice following both acute (I) and 24-hour (J) feeding assays. Data represents mean+/−s.e.m. Panels A, D, F, and I analyzed by 2-way ANOVA with Tukey's post hoc test. Panels B, C, G, H, and J analyzed with unpaired Student's t-test. n=7 mice and n=8 mice for vehicle and C18 groups in panels A-C. n=19 mice for panels F and G. n=6 mice for vehicle and n=8 mice for C11 group in panel H. n=12 mice for vehicle and C18 group in panels D and E. n=8 mice for vehicle and C11 groups in panels I and J.

FIG. 29A-E. MC3R specific compounds regulate feeding via AgRP circuitry. A and B: Schematic of experimental strategy to express inhibitory DREADD virus in hypothalamic AgRP neurons (A) and representative image of DREADD transduced AgRP neurons (B). Scale bar, 200 um. C: Food intake following administration of C18 in the presence of saline or CNO (0.1 mg/kg, i.p.). C18 increased food intake following administration of saline but not following CNO mediated inhibition of AgRP neurons. D: RNAscope analysis of MC3R expression in WT and AgRP-MC3R KI mice. MC3R expression is observed in multiple ARH cells types and in the paraventricular thalamus (PVT) in WT mice, while expression is only observed in AgRP neurons in the AgRP-MC3R KI mice. Scale bar, 500 um. E: Food intake (top panel) and change in body weight (bottom panel) 24 hours following administration of vehicle of C11 to WT and AgRP-MC3R knockin mice. Food intake and body weight was significantly reduced following C11 administration in AgRP-MC3R knockin mice. Panel C analyzed by 2-way ANOVA with Tukey's post hoc test. Panel E (top panel) analyzed by paired Student's t-test or unpaired Student's t-test (bottom panel). n=20 mice for vehicle saline group, n=12 mice for vehicle C18 group, n=9 mice for CNO vehicle group, and n=15 mice for CNO C18 group in panel C. n=8 mice for AgRP-MC3R KI group in panel E. *p<0.05, **p<0.01.

FIG. 30A-I. MC3R agonism inhibits PVN MC4R neurons in vivo. A and B: Representative image depicting miniaturized microscope used for in vivo imaging studies (A) and viral injection paradigm used to express the genetically encoded calcium indicator GCAMP6s in PVN MC4R neurons (B).C: Representative image showing expression of GCAMP6s in PVN MC4R neurons and lens placement in the PVN. Scale bar, 500 um. D: Change in florescence following i.p. administration of saline. No change in florescence was detected following saline administration. E: 4-hour food intake in WT mice following administration of the MC3R agonist C18 (i.p., 10 mg/kg). C18 increased food intake in WT mice. F: Change in fluorescence following i.p. administration of the MC3R agonist C18 (10 mg/kg, i.p.). Fluorescence activity was reduced in PVN MC4R neurons following administration of C18. G: Heat plot showing real-time change in fluorescence activity in a representative mouse during baseline and following administration of C18. H: Average trace of the relative change in fluorescence before and after C18 administration. I: Food intake following administration of vehicle (aCSF) or C18 (5 ug, 300 nl) into PVN. PVN administration of C18 increased food intake relative to vehicle treatment conditions. Data represents mean+/−s.e.m. for panels D-F. Panels D, F, and I analyzed with paired Student's t-test. Panel E analyzed with Student's unpaired t-test. n=3 mice and 31 neurons for panel D. n=3 mice and 32 neurons for panel F. n=8 mice for vehicle group and n=7 mice for C18 group in panel E. n=7 mice for panel I. *p<0.05, ****p<0.001.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “an MC3R agonist” is a reference to one or more MC3R agonists and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “MC3R agonist” refers to an agent (e.g., small molecule, peptide, etc.) that binds to MC3R and activates MC3R to produce its biological activity. In some embodiments, an MC3R agonist binds to MC3R in the same location as a natural MC3R ligand (e.g., melanocyte-stimulating hormone and adrenocorticotropic hormone) and produce a functional response.

As used herein, the term “MC3R antagonist” refers to an agent (e.g., small molecule, peptide, etc.) that binds to MC3R and inhibits MC3R's biological activity. In some embodiments, an MC3R antagonist is a competitive antagonist and binds to MC3R in the same location as a natural MC3R ligand (e.g., melanocyte-stimulating hormone and adrenocorticotropic hormone) and inhibits binding of the natural ligand to the receptor. In some embodiments, an MC3R antagonist is a non-competitive antagonist and binds to MC3R in a distinct location from a natural MC3R ligand (e.g., melanocyte-stimulating hormone) but still inhibits the biological activity of MC3R.

As used herein, the term “MC3R inhibitor” refers to an agent (e.g., small molecule, peptide, antibody, antibody fragment, aptamer, nucleic acid, etc.) that reduces MC3R activity of expression. An MC3R inhibitor may function by any suitable mechanism, including but not limited to reducing/inhibiting expression of MC3R (e.g., RNAi, antisense RNA, etc.), sequestering MC3R (e.g., antibody), preventing interaction of MC3R with other components involved in its function (e.g. G-protein), etc.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “anorexia nervosa” or synonymously “anorexia” a psychological condition characterized by a relentless desire to lose weight in the pursuit of thinness to the point of cachexia by voluntarily withholding foods and fluids, and, at times, by excessive exercising.

As used herein, the term “subject at risk for a disease,” for example, “a subject at risk for anorexia” or “a subject at risk for anxiety” refers to a subject with one or more risk factors for developing the disease (e.g., cancer). Depending upon the specific disease, risk factors may include, but are not limited to, gender, age, genetic predisposition, environmental exposures, infections, and previous incidents of diseases, lifestyle, etc.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., an MC3R agonist or antagonist and one or more additional therapeutics) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

As used herein, the term “instructions for administering said compound to a subject,” and grammatical equivalents thereof, includes instructions for using the compositions contained in a kit for the treatment of conditions (e.g., providing dosing, route of administration, decision trees for treating physicians for correlating patient-specific characteristics with therapeutic courses of action).

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “Pgly”), pipecolic acid, thioproline (“ThioP” or “tPro”), homoLysine (“hLys”), and iethyldithi (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 30 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “artificial” refers to compositions and systems that are designed or prepared by man, and are not naturally occurring. For example, an artificial peptide, peptoid, or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:

1) Alanine (A) and Glycine (G);

2) Aspartic acid (D) and Glutamic acid I;

3) Asparagine (N) and Glutamine (Q);

4) Arginine I and Lysine (K);

5) Isoleucine (I), Leucine (L), Methionine (M), and Valine (V);

6) Phenylalanine (F), Tyrosine (Y), and Tryptophan (W);

7) Serine (S) and Threonine (T); and

8) Cysteine I and Methionine (M).

Naturally occurring residues may be divided into classes based on common side chain properties, for example: polar positive (or basic) (histidine (H), lysine (K), and arginine I); polar negative (or acidic) (aspartic acid (D), glutamic acid I); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs. Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y% sequence identity (e.g., 90%) with SEQ ID NO:Z (e.g., 20 amino acids) may have up to X substitutions (e.g., 2) relative to SEQ ID NO:Z, and may therefore also be expressed as “having X (e.g., 2) or fewer substitutions relative to SEQ ID NO:Z.”

As used herein, the term “antibody” refers to a whole antibody molecule or a fragment thereof (e.g., fragments such as Fab, Fab′, and F(ab′)₂), it may be a polyclonal or monoclonal antibody, a chimeric antibody, a humanized antibody, a human antibody, etc.

A native antibody typically has a tetrameric structure. A tetramer typically comprises two identical pairs of polypeptide chains, each pair having one light chain (in certain embodiments, about 25 kDa) and one heavy chain (in certain embodiments, about 50-70 kDa). In a native antibody, a heavy chain comprises a variable region, VH, and three constant regions, C_{H1}, C_{H2}, and C_{H3}. The V_{H} domain is at the amino-terminus of the heavy chain, and the C_{H3} domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and 1-R4. The CDRs on the heavy chain are referred to as H1, H2, and H3, while the CDRs on the light chain are referred to as L1, L2, and L3. Typically, CDR3 is the greatest source of molecular diversity within the antigen-binding site. H3, for example, in certain instances, can be as short as two amino acid residues or greater than 26. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol. 196:901-917; or Chothia, C. et al. Nature 342:878-883 (1989). In the present application, the term “CDR” refers to a CDR from either the light or heavy chain, unless otherwise specified.

As used herein, the terms “anti-MC3R antibody” or “MC3R antibody” refer to an antibody which specifically recognizes an antigen and/or epitope presented by MC3R.

As used herein, the term “monoclonal antibody” refers to an antibody which is a member of a substantially homogeneous population of antibodies that specifically bind to the same epitope. In certain embodiments, a monoclonal antibody is secreted by a hybridoma. In certain such embodiments, a hybridoma is produced according to certain methods known to those skilled in the art. See, e.g., Kohler and Milstein (1975) Nature 256: 495-499; herein incorporated by reference in its entirety. In certain embodiments, a monoclonal antibody is produced using recombinant DNA methods (see, e.g., U.S. Pat. No. 4,816,567). In certain embodiments, a monoclonal antibody refers to an antibody fragment isolated from a phage display library. See, e.g., Clackson et al. (1991) Nature 352: 624-628; and Marks et al. (1991) J. Mol. Biol. 222: 581-597; herein incorporated by reference in their entireties. The modifying word “monoclonal” indicates properties of antibodies obtained from a substantially-homogeneous population of antibodies, and does not limit a method of producing antibodies to a specific method. For various other monoclonal antibody production techniques, see, e.g., Harlow and Lane (1988) Antibodies: A Laboratory Manual (Cold Spring Harbor Laboratory, Cold Spring Harbor, N.Y.); herein incorporated by reference in its entirety.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis.

For example, a “Fab” fragment comprises one light chain and the C_{H1} and variable region of one heavy chain. The heavy chain of a Fab molecule cannot form a disulfide bond with another heavy chain molecule. A “Fab” fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule.

An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen.

Other antibody fragments will be understood by skilled artisans.

### DETAILED DESCRIPTION

Provided herein are compositions and methods for targeting (e.g., inhibiting or enhancing the activity or expression of) melanocortin 3 receptor (MC3R) gene, mRNA, and protein for the treatment and/or prevention of eating disorders (e.g., anorexia nervosa, cachexia, etc.), metabolic disorders (e.g., obesity, diabetes, non-alcoholic steatohepatitis, hypertension, etc.), and/or emotional/mental disorders (e.g., depression, anxiety, OCD, PTSD, etc.). In particular, provided herein are MC3R agonists that stimulate MC3R for the treatment/prevention of disorders such as anorexia nervosa and other eating and/or anxiety disorders, MC3R antagonists that inhibit MC3R for the treatment/prevention of obesity and/or other eating/metabolism disorders, and methods of use thereof.

Experiments conducted during development of embodiments herein demonstrate that MC3R agonists stimulate feeding, increase body weight, and reduce anxiety in an AgRP neuron dependent manner Experiments show that MC3R is highly expressed in arcuate AgRP neurons, with significantly higher expression in these cells than anorexigenic POMC neurons. MC3R agonist treatment phenocopies chemogenetic or optogenetic activation of ARC MC3R neurons, both stimulating feeding and body weight and reducing anxiety-related behavior. Conversely, chemogenetic inhibition of these cells reduces feeding and increases anxiety-related behavior. Experiments conducted during development of embodiments herein demonstrate that test subjects lacking the MC3R display multiple behavioral phenotypes resembling anorexia nervosa, such as enhanced anxiety behavior and increased susceptibility to multiple forms of stress-induced anorexia. Experiments conducted during development of embodiments herein indicate that stimulation of the MC3R is a therapeutic approach for combating disorders at the intersection of energy metabolism and emotion, such as anorexia nervosa.

Central regulation of feeding and body weight is primarily controlled by neural circuits located in the hypothalamus and hindbrain (Refs. 1-3; herein incorporated by reference in their entireties). The central melanocortin system, composed of a set of two neuronal cell types located in the hypothalamic arcuate nucleus, the agouti related peptide neurons (AgRP neurons) and the pro-opiomelanocortin neurons (POMC neurons), engages this hypothalamic and hindbrain circuitry to potently regulate feeding and body weight (Refs. 4-6; herein incorporated by reference in their entireties). AgRP and POMC neurons project to largely overlapping brain regions to exert opposing effects on feeding and body weight. For example, AgRP neurons synthesize and release the melanocortin receptor antagonist/inverse agonist, agouti related peptide (AgRP), GABA, and neuropeptide Y to stimulate feeding and body weight (Ref. 7; herein incorporated by reference in its entirety). In contrast, POMC neurons synthesize and release the endogenous melanocortin receptor agonist, alpha melanocyte stimulating hormone (α-MSH), in addition to fast excitatory/inhibitory neurotransmitters to suppress feeding and reduce body weight (Refs. 4, 8; herein incorporated by reference in their entireties).

Recent work has highlighted the neural circuits and mechanisms governing the potent role of hypothalamic AgRP neurons in stimulating feeding (Refs. 6, 9, 10; herein incorporated by reference in their entireties). Ablation of AgRP neurons in adult mice leads to starvation and death, while stimulation rapidly and robustly stimulates food intake and body weight in sated animals (Refs. 11-13; herein incorporated by reference in their entireties). In addition to stimulating feeding, AgRP neuronal activation also suppresses competing need states, such as anxiety and fear, thereby promoting food seeking behavior in response to negative energy balance (Refs. 14-15; herein incorporated by reference in their entireties). Intense effort has focused on identifying pharmacological targets which suppress AgRP neural circuits as a potential therapeutic strategy for obesity.

MC3R is a G-protein coupled receptor primarily expressed within the brain, with particular dense expression observed in the hypothalamic arcuate nucleus (Refs. 16-17; herein incorporated by reference in their entireties). MC3R is expressed in AgRP neurons and recent studies suggest that MC3R has an important role in regulating the orexigenic activity of these cells (Ref. 16; herein incorporated by reference in its entirety). For example, MC3R knockout mice show multiple deficits in conditions that activate AgRP neurons, such as impaired feeding in response to a fast or caloric restriction (Refs. 18-20; herein incorporated by reference in their entireties). MC3R acts within presynaptic AgRP terminals in the paraventricular hypothalamus (PVN), promoting GABA release onto anorexigenic PVN melanocortin 4 receptor expressing neurons (Ref. 18; herein incorporated by reference in its entirety).

Experiments conducted during development of embodiments herein demonstrate that the MC3R is broadly distributed within the hypothalamic arcuate nucleus, with particularly enriched expression in the orexigeneic AgRP neurons. Multiple lines of experimentation demonstrate that elective chemogenetic activation of all arcuate neurons expressing MC3R profoundly stimulates food intake and body weight, while reducing anxiety. Conversely, these experiments demonstrate that inhibition of ARC-MC3R neurons produces the opposite effect, reducing feeding and body weight, and increasing anxiety. Treatment with selective MC3R agonists recapitulates the effects observed following activation of ARC-MC3R neurons (by DREADDs; Designer Receptors Exclusively Activated by Designer Drugs), potently stimulating food intake, increasing body weight, and reducing anxiety behavior. Experiments conducted during development of embodiments herein demonstrate that test subjects lacking the MC3R display multiple behavioral phenotypes resembling anorexia nervosa, such as enhanced anxiety and hyper-sensitivity to stress-induced anorexia. These findings indicate that pharmacological therapies targeting the MC3R have utility in treating both the neuropsychiatric and metabolic phenotypes associated with anorexia nervosa and other diseases of negative energy balance.

Accordingly, embodiments herein provide for the modulation (e.g., inhibition or activation) of MC3R in order to achieve a desired impact on an eating disorder (e.g., anorexia, orthorexia, cachexia, etc.), eating habits (e.g., overeating, undereating, etc.), weight (e.g., obesity), a psychological condition (e.g., anxiety, depression, etc.), etc. In some embodiments, an agent for inhibiting (e.g., MC3R antagonist) or activating (e.g., MC3R agonist) MC3R are administered to a subject and/or co-administered with one or more additional therapeutics/therapies.

### I. MC3R Agonism

Experiments conducted during development of embodiments herein demonstrate that administration of MC3R agonists is effective in the treatment and/or prevention of various eating disorders (e.g., anorexia nervosa, cachexia, etc.), metabolic disorders, and/or emotional/mental disorders (e.g., depression, anxiety, etc.). In some embodiments, provided herein are methods of treating, preventing, and/or ameliorating the symptoms of eating disorders (e.g., anorexia nervosa, cachexia, etc.), metabolic disorders, and/or emotional/mental disorders (e.g., depression, anxiety, etc.) by enhancing of the activity and/or expression of MC3R in a subject.

In some embodiments, the subject suffers from an eating disorder such as anorexia nervosa, bulimia nervosa, pica, rumination disorder, avoidant or restrictive food intake disorder, orthorexia nervosa, etc. In some embodiments, the subject suffers from anorexia. In some embodiments, the subject is at risk of developing an eating disorder (e.g., anorexia), having a recurrence of an eating disorder, relapsing into an eating disorder, or exhibiting physical sympotoms of an eating disorder (e.g., low weight, restrictive eating, weight loss, etc.).

In some embodiments, the subject suffers from a psychological condition or mental illness such as anxiety depression, dipolar affective disorder, psychoses, obsessive compulsive disorder, post-traumatic stress disorder, etc. In some embodiments, the subject suffers from anxiety. In some embodiments, the subject is at risk of developing mental illness or exhibiting symptoms of a mental illness.

In some embodiments, an MC3R agonist is administered to a subject (e.g., by any suitable route of administration and within any suitable pharmaceutical formulation). In some embodiments, the MC3R agonist bind to MC3R in the subject. In some embodiments, the activity of MC3R is enhanced by the administration of an MC3R agonist.

In some embodiments, methods herein comprise administering an MC3R agonist to a subject at risk of and/or suffering from an eating disorder (e.g., anorexia) and/or a mental illness (e.g., anxiety). In some embodiments, administration of the MC3R agonist results in increased eating, bodyweight, and/or reduced anxiety in the subject.

In some embodiments, the MC3R agonist is administered locally. In some embodiments, the MC3R agonist is administered systemically. In some embodiments, the MC3R agonist is administered in a manner such that the MC3R agonist reaches and/or localizes in the brain. In some embodiments, the MC3R agonist is administered in a manner such that the MC3R agonist reaches and/or localizes in the hypothalamus. In some embodiments, the MC3R agonist is administered in a manner such that the MC3R agonist reaches and/or localizes in AgRP neurons. In some embodiments, the MC3R agonist is administered in a manner such that the MC3R agonist reaches and/or localizes in POMC neurons.

In some embodiments, an MC3R agonist is a small molecule drug. Exemplary small molecule MC3R agonists include those identified in the small molecule screen described in Example 2. Any suitable MC3R agonists identified in a small molecule screen described herein (e.g., as in Example 2) or understood elsewhere may find use in embodiments herein.

In some embodiments, suitable MC3R agonists are described in, for example Bednarek, et al., Peptides 28 (2007) 1020-1028; Carotenutoet al., J. Med. Chem. 58 (2015) 9773-9778.; Ericson et al. Molecular Basis of Disease 1863 (2017) 2414-2435; incorporated by reference in their entireties. For example:

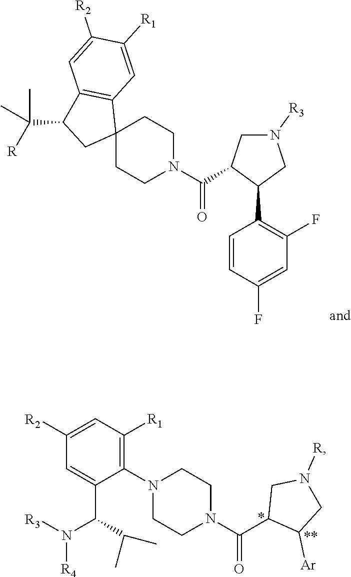

as described in Bednarek, et al., Carotenutoet al., and/or Ericson et al.

In some embodiments, an MC3R agonist is a peptide, such as the peptide agonists depicted in Table 2 (e.g., D-Trp8-γMSH, PG990, PG992, or FMMC-5), or dep. In some embodiments, an MC3R peptide agonist comprises at least 50% sequence identity (e.g., >50%, >60%, >70%, >80%, >90%, 100%) with SEQ ID NO: 1 (D-Trp8-γMSH). In some embodiments, an MC3R peptide agonist comprises at least 50% sequence identity (e.g., >50%, >60%, >70%, >80%, >90%, 100%) with SEQ ID NO: 2 (PG990; Ac-Nle-c[Asp-Pro-Pro-DNal(2)-Arg-Trp-Lys]-NH₂). In some embodiments, an MC3R peptide agonist comprises at least 50% sequence identity (e.g., >50%, >60%, >70%, >80%, >90%, 100%) with SEQ ID NO: 3 (PG992; Ac-Nle-c[Asp-Trp-Pro-DNal(2)-Arg-Trp-Lys]-NH2). In some embodiments, an MC3R peptide agonist comprises at least 50% sequence identity (e.g., >50%, >60%, >70%, >80%, >90%, 100%) with FMMC-5.

In some embodiments, an MC3R peptide agonist comprises a HFRW (SEQ ID NO: 5) tetrapeptide sequence. In some embodiments, an MC3R peptide agonist comprises at least 50% sequence identity (e.g., >50%, >60%, >70%, >80%, >90%, 100%) with SEQ ID NO: 1, and comprises the HFRW (SEQ ID NO: 5) tetrapeptide sequence. In some embodiments, an MC3R peptide agonist comprises at least 50% sequence identity (e.g., >50%, >60%, >70%, >80%, >90%, 100%) with FMMC-5, and comprises the HFRW (SEQ ID NO: 5) tetrapeptide sequence.

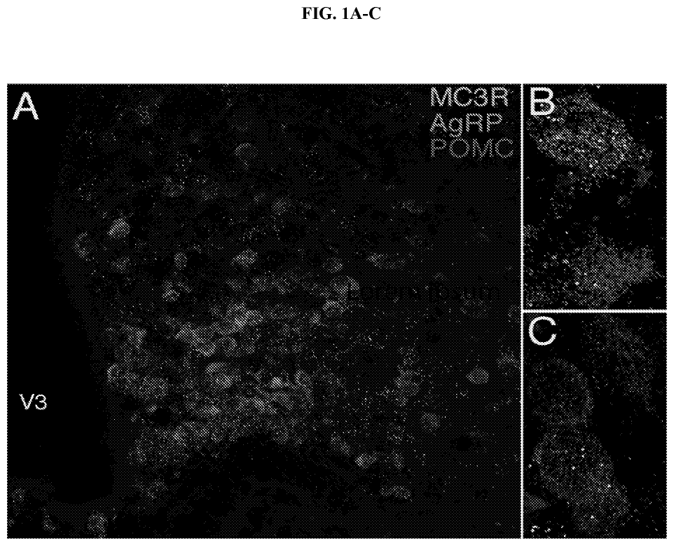

In some embodiments, MC3R agonists are described in Ericson et al. Molecular Basis of Disease 1863 (2017) 2414-2435; herein incorporated by reference in its entirety. Any suitable agonists or other molecules described therein are included within the scope of this disclosure. For example, FIG. 26 (reproduced from FIG. 1A of Ericson et al.) depicts SEQ ID NOS: 6-11, POMC-derived naturally-occurring melancortin agonist peptides. In some embodiments, a peptide comprising one or SEQ ID NO: 6, 7, 8, 9, 10, or 11, or a variant comprising at least 50% (e.g., >50%, >60%, >70%, >80%, >90%, 100%) sequence identity therewith, may find use in embodiments herein. In some embodiments, an MC3R peptide agonist comprises at least 50% sequence identity (e.g., >50%, >60%, >70%, >80%, >90%, 100%) with one of SEQ ID NO: 6, 7, 8, 9, 10, or 11, and comprises the HFRW (SEQ ID NO: 5) tetrapeptide sequence.

In some embodiments, a MC3R agonist is comprises or is a variant of the tetrapeptide scaffold Ac-Xaa¹-Arg-(pI)DPhe-Xaa⁴-NH₂ (SEQ ID NO: 12) (Doering et al. Journal of Medicinal Chemistry vol. 60,10 (2017); incorporated by reference in its entirety). In some embodiments, Xaa¹ and Xaa⁴ are selected from Table 3.

TABLE 3
Tetrapeptide MC3R agonists

| Peptide # | Xaa¹ | Xaa4 |
|---|---|---|
| 1 | His | Tic |
| 2 | Arg | Bip |
| 3 | His | Bip |
| 4 | Bip | Bip |
| 5 | 3Bal | Bip |
| 6 | Tic | Bip |
| 7 | Phe | Bip |
| 8 | Nal(2′) | Bip |
| 9 | DNal(2′) | Bip |
| 10 | Arg | 3Bal |
| 11 | His | 3Bal |
| 12 | Bip | 3Bal |
| 13 | 3Bal | 3Bal |
| 14 | Tic | 3Bal |
| 15 | Phe | 3Bal |
| 16 | Nal(2′) | 3Bal |
| 17 | DNal(2′) | 3Bal |
| 18 | Arg | Tic |
| 19 | Bip | Tic |
| 20 | 3Bal | Tic |
| 21 | Tic | Tic |
| 22 | Phe | Tic |
| 23 | Nal(2′) | Tic |
| 24 | DNal(2′) | Tic |
| 25 | Arg | Phe |
| 26 | His | Phe |
| 27 | Bip | Phe |
| 28 | 3Bal | Phe |
| 29 | Tic | Phe |
| 30 | Phe | Phe |
| 31 | Nal(2′) | Phe |
| 32 | DNal(2′) | Phe |
| 33 | Arg | Nal(2′) |
| 34 | His | Nal(2′) |
| 35 | Bip | Nal(2′) |
| 36 | 3Bal | Nal(2′) |
| 37 | Tic | Nal(2′) |
| 38 | Phe | Nal(2′) |
| 39 | Nal(2′) | Nal(2′) |
| 40 | DNal(2′) | Nal(2′) |
| 41 | Arg | DNal(2′) |
| 42 | His | DNal(2′) |
| 43 | Bip | DNal(2′) |
| 44 | 3Bal | DNal(2′) |
| 45 | Tic | DNal(2′) |
| 46 | Phe | DNal(2′) |
| 47 | Nal(2′) | DNal(2′) |
| 48 | DNal(2′) | DNal(2′) |

In some embodiments, an MC3R agonist is compound 18 (Ac-Arg-Arg-(pI)DPhe-Tic-NH2; SEQ ID NO: 13), compound 1 (Ac-His-Arg-(pI)DPhe-Tic-NH2; SEQ ID NO: 14), and compound 41 (Ac-Arg-Arg-(pI)DPhe-DNa1(2′)-NH2; SEQ ID NO: 15).

In some embodiments, an MC3R agonist binds MC3R selectively over other melanocortin receptors (e.g., MC1R, MC2R, MC4R, MC5R). In some embodiments, an MC3R agonist binds MC3R with an affinity that is at least 2-fold greater (e.g., 2×, 3×, 4×, 5×, 6×, 7×, 8×, 9×, 10×, 20×, 30×, 40×, 50×, 60×, 70×, 80×, 90×, 100×, or more) than the binding affinity of the MC3R agonist with other melanocortin receptors (e.g., MC1R, MC2R, MC4R, MC5R). In some embodiments, an MC3R agonist binds MC3R selectively over MC4R. In some embodiments, an MC3R agonist binds MC3R with an affinity that is at least 2-fold greater (e.g., 2×, 3×, 4×, 5×, 6×, 7×, 8×, 9×, 10×, 20×, 30×, 40×, 50×, 60×, 70×, 80×, 90×, 100×, or more) than the binding affinity of the MC3R agonist with MC4R.

In some embodiments, an MC3R agonist enhances the activity of MC3R selectively over other melanocortin receptors (e.g., MC1R, MC2R, MC4R, MC5R). In some embodiments, an MC3R agonist enhances the activity of MC3R at least 2-fold greater (e.g., 2×, 3×, 4×, 5×, 6×, 7×, 8×, 9×, 10×, 20×, 30×, 40×, 50×, 60×, 70×, 80×, 90×, 100×, or more) than other melanocortin receptors (e.g., MC1R, MC2R, MC4R, MC5R). In some embodiments, an MC3R agonist enhances the activity of MC3R selectively over MC4R. In some embodiments, an MC3R agonist enhances the activity of MC3R at least 2-fold greater (e.g., 2×, 3×, 4×, 5×, 6×, 7×, 8×, 9×, 10×, 20×, 30×, 40×, 50×, 60×, 70×, 80×, 90×, 100×, or more) than MC4R.

In some embodiments, an MC3R agonist is co-administered with an additional agent or therapy. In some embodiments, the co-administered agent is for the treatment or prevention of the same condition/disease/symptom as the MC3R agonist (e.g., anorexia, anxiety, etc.). In some embodiments, the co-administered agent is for the treatment or prevention of a side-effect of the MC3R agonist. In some embodiments, the co-administered agent is for the treatment or prevention of a comorbidity not treated of prevented by the MC3R agonist (e.g., bulimia, depression, obsessive-compulsive disorder, pain, etc.).

In some embodiments, the MC3R agonist is co-administered with psychotherapy, nasogastric feeding, antidepressant agents, antianxiety agents, mood stabilizers, stimulants, and/or antipsychotic agents.

The term “psychotherapy” refers to use of non-pharmacological therapies a clinician or therapist uses any of a variety of techniques that involve verbal and other interactions with a patient to affect a positive therapeutic outcome. Such techniques include, but are not limited to, behavior therapy, cognitive therapy, psychodynamic therapy, psychoanalytic therapy, group therapy, family counseling, art therapy, music therapy, vocational therapy, humanistic therapy, existential therapy, transpersonal therapy, client-centered therapy (also called person-centered therapy), Gestalt therapy, biofeedback therapy, rational emotive behavioral therapy, reality therapy, response based therapy, Sandplay therapy, status dynamics therapy, hypnosis and validation therapy. Any suitable psychotherapy techniques, including those listed above, may be co-administered with an MC3R agonist for the treatment/prevention of appropriate conditions/diseases (e.g., eating disorders (e.g., anorexia, cachexia, etc.), mental disease (e.g., anxiety, depression, etc.), etc.

Nasogastric (NG) feeding involves the use of a special tube (NG tube) that carries food to the stomach through the nose. In some embodiments, NG feeding is utilized in place of oral feeding and/or as a supplement to oral feeding, particularly in the earliest stage of treatment for an eating disorder. In some embodiments, NG feeding is co-administered with an MC3R agonist. In some embodiments, NG feeding is ceased once eating orally becomes sufficient to produce weight gain (e.g., as a result of the effect of the MC3R agonist).

In some embodiments, an MC3R agonist is co-administered with an antidepressant agent. Suitable antidepressants for co-administration may include serotonin and noradrenaline reuptake inhibitors (e.g., duloxetine (Cymbalta), venlafaxine (Effexor), desvenlafaxine (Pristiq), etc.), selective serotonin reuptake inhibitors (e.g., italopram (Celexa), escitalopram (Lexapro), fluoxetine (Prozac, Sarafem), fluvoxamine (Luvox), paroxetine (Paxil), sertraline (Zoloft), etc.), tricyclic antidepressants (e.g., amitriptyline (Elavil), amoxapine- clomipramine (Anafranil), desipramine (Norpramin), doxepin (Sinequan), imipramine (Tofranil), nortriptyline (Pamelor), protriptyline (Vivactil), trimipramine (Surmontil), etc.), monoamine oxidase inhibitors (e.g., phenelzine (Nardil), tranylcypromine (Parnate), isocarboxazid (Marplan), selegiline (EMSAM, Eldepryl), etc.), noradrenaline and specific serotoninergic antidepressants (e.g., Mianserin (Tolvon), Mirtazapine (Remeron, Avanza, Zispin, etc.), etc.

In some embodiments, an MC3R agonist is co-administered with an antianxiety agent. Suitable antianxiety medications for co-administration may include selective serotonin reuptake inhibitors, serotonin-norepinephrine reuptake inhibitors, tricyclics, benzodiazepines (e.g., alprazolam (Xanax), chlordiazepoxide (Librium), diazepam (Valium), lorazepam (Ativan) etc.), beta-blockers (e.g., atenolol (Tenormin), propranolol (Inderal), etc.), buspirone (BuSpar), monoamine oxidase inhibitors, etc.

In some embodiments, an MC3R agonist is co-administered with a mood stabilizer. Suitable mood stabilizers for co-administration may include lithium, anticonvulsants (e.g., valproate, lamotrigine, carbamazepine, etc.), etc.

In some embodiments, an MC3R agonist is co-administered with a stimulant. Suitable stimulants for co-administration may include amphetamine/dextroamphetamine (Adderall), dextroamphetamine (Dexedrine, ProCentra, Zenzedi), dexmethylphenidate (Focalin), methylphenidate (Ritalin), amphetamine sulfate (Evekeo), methylphenidate (Ritalin SR, Metadate ER, Methylin ER), amphetamine (Adzenys XR-ODT, Dyanavel XR), dexmethylphenidate (Focalin XR), dextroamphetamine (Adderall XR), lisdexamfetamine (Vyvanse), methylphenidate (Concerta, Daytrana, Jornay PM, Metadate CD, Quillivant XR, Quillichew ER, Ritalin LA), etc.

In some embodiments, an MC3R agonist is co-administered with any agent or medication suitable for the treatment of the eating disorders and/or mental illnesses described herein.

### II. MC3R Antagonism/Inhibition

Experiments conducted during development of embodiments herein demonstrate that administration of MC3R antagonists is effective in the treatment and/or prevention of various conditions related to overeating (e.g., obesity). In some embodiments, provided herein are methods of treating, preventing, and/or ameliorating the symptoms of overeating (e.g., obesity) by inhibiting of the activity and/or expression of MC3R in a subject.

In some embodiments, the subject suffers from obesity, diabetes, heart disease, hypertension, sleep apnea, depression, kidney disease, arthritis, etc. In some embodiments, the subject suffers from obesity. In some embodiments, the subject is at risk of overeating or becoming obese. In some embodiments, the subject has recovered from obesity or an over-eating disorder and is at risk of relapsing.

Accordingly, in some embodiments, provided herein are methods of treating, preventing, and/or ameliorating the symptoms of obesity and/or overeating by inhibition of the activity or expression of MC3R. Diseases and conditions that are addressed (e.g., treated, prevented, ameliorated, etc.) in embodiments herein include and diseases/conditions/symptoms in which overeating and/or obesity is a cause, contributing factor, or risk factor. Such diseases/conditions/symptoms include, but are not limited to heart disease, diabetes, cancer, arthritis, gout, asthma, sleep apnea, hypertension, stroke, etc.

In some embodiments, an antagonist/inhibitor of the activity of MC3R is administered to a subject (e.g., by any suitable route of administration and within any suitable pharmaceutical formulation). In some embodiments, expression of MC3R is inhibited (e.g., partially or completely), for example, by siRNA, or genetic manipulation (e.g., by CRISPR).

In some embodiments, methods herein comprise administering an MC3R antagonist to a subject at risk of obesity and/or suffering from obesity or an overeating disorder.

In some embodiments, an MC3R antagonist is a small molecule drug. Exemplary small molecule MC3R antagonists include any identified in a small molecule screen of MC3R antagonists (e.g., the screen of Example 2), such as: polymyxin B, VU0658058, VU0657981, VU0658096, VU0657988, VU0658085 (See, e.g., FIG. 19B).

In some embodiments, an MC3R antagonist is a peptide.

In some embodiments, the technology provides a method for inhibiting MC3R activity by administering an antibody or fragment that recognizes, binds, and inhibits the activity of MC3R. In some embodiments, the antibody is a monoclonal antibody and in some embodiments the antibody is a polyclonal antibody. In some embodiments, the antibody is, for example, a human, humanized, or chimeric antibody. Monoclonal antibodies against target antigens are produced by a variety of techniques including conventional monoclonal antibody methodologies such as the somatic cell hybridization techniques of Köhler and Milstein (Nature, 256:495 (1975)). Although in some embodiments, somatic cell hybridization procedures are preferred, other techniques for producing monoclonal antibodies are contemplated as well.

In some embodiments, methods herein comprise inhibiting the expression of MC3R. Multiple methods of altering gene expression within a cell, tissue, or subject are known in the field (e.g., RNAi, antisense RNA, gene therapy, CRISPR, etc.).

In some embodiments, a nucleic acid is used to modulate expression of MC3R.

For example, in some embodiments a small interfering RNA (siRNA) is designed to target and degrade MC3R. siRNAs are double-stranded RNA molecules of 20-25 nucleotides in length. While not limited in their features, typically an siRNA is 21 nucleotides long and has 2-nt 3′ overhangs on both ends. Each strand has a 5′ phosphate group and a 3′ hydroxyl group. In vivo, this structure is the result of processing by Dicer, an enzyme that converts either long dsRNAs or small hairpin RNAs (shRNAs) into siRNAs. However, siRNAs can also be synthesized and exogenously introduced into cells to bring about the specific knockdown of a gene of interest. Essentially any gene of which the sequence is known can be targeted based on sequence complementarity with an appropriately tailored siRNA. For example, those of ordinary skill in the art can synthesize an siRNA (see, e.g., Elbashir, et al.,

Nature 411: 494 (2001); Elbashir, et al. Genes Dev 15 :188 (2001); Tuschl T, et al., Genes Dev 13 :3191 (1999); incorporated by reference in their entireties).

In some embodiments, RNAi is utilized to inhibit expression of MC3R. In some embodiments, RNAi is used to modulate expression of MC3R. RNAi represents an evolutionarily conserved cellular defense for controlling the expression of foreign genes in most eukaryotes, including humans. RNAi is typically triggered by double-stranded RNA (dsRNA) and causes sequence-specific degradation of single-stranded target RNAs (e.g., an mRNA). The mediators of mRNA degradation are small interfering RNAs (siRNAs), which are normally produced from long dsRNA by enzymatic cleavage in the cell. siRNAs are generally approximately twenty-one nucleotides in length (e.g. 21-23 nucleotides in length) and have a base-paired structure characterized by two-nucleotide 3′ overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, an RNase III enzyme (e.g., Dicer) converts the longer dsRNA into 21-23 nt double-stranded siRNA fragments. In some embodiments, RNAi oligonucleotides are designed to target the junction region of fusion proteins. Chemically synthesized siRNAs have become powerful reagents for genome-wide analysis of mammalian gene function in cultured somatic cells. Beyond their value for validation of gene function, siRNAs also hold great potential as gene-specific therapeutic agents (see, e.g., Tuschl and Borkhardt, Molecular Intervent. 2002; 2(3): 158-67, herein incorporated by reference).

In other embodiments, shRNA techniques (See e.g., 20080025958, herein incorporated by reference in its entirety) are utilized to modulate (e.g., inhibit) expression of MC3R. A small hairpin RNA or short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA that is bound to it. shRNA is transcribed by RNA polymerase III.

In some embodiments, an antisense nucleic acid (e.g., an antisense DNA oligo, an antisense RNA oligo) is used to modulate the expression of MC3R. For example, in some embodiments, expression of MC3R is inhibited using antisense compounds that specifically hybridize with nucleic acids MC3R. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation (e.g., inhibition) of the expression of MC3R.

In some embodiments, MC3R activity and/or expression are inhibited using the CRISPR/Cas system. “CRISPRs” (clustered regularly interspaced short palindromic repeats), as described herein, are segments of prokaryotic DNA containing short repetitions of base sequences. Each repetition is followed by short segments of “spacer DNA” from previous exposures to a bacterial virus or plasmid. The CRISPR/Cas system is a prokaryotic immune system that confers resistance to foreign genetic elements such as plasmids and phages and provides a form of acquired immunity. CRISPR spacers recognize and cut these exogenous genetic elements in a manner analogous to RNAi in eukaryotic organisms. CRISPR/Cas system has been used for gene editing (adding, disrupting or changing the sequence of specific genes) and gene regulation in species throughout the tree of life. By delivering the Cas9 protein and appropriate guide RNAs into a cell, the organism's genome can be cut at any desired location. One can use CRISPR to build RNA-guided gene editing tools capable of altering the genome of a subject. In some embodiments, the CRISPR/Cas system is utilized to inhibit (e.g., partially or completely) the expression of MC3R in a subject, tissue, or cells. In some embodiments, the CRISPR/Cas system is utilized to produce MC3R that is of reduced activity (e.g., in a subject, tissue, or cells.

In some embodiments, an MC3R antagonist binds MC3R selectively over other melanocortin receptors (e.g., MC1R, MC2R, MC4R, MC5R). In some embodiments, an MC3R antagonist binds MC3R with an affinity that is at least 2-fold greater (e.g., 2×, 3×, 4×, 5×, 6×, 7×, 8×, 9×, 10×, 20×, 30×, 40×, 50×, 60×, 70×, 80×, 90×, 100×, or more) than the binding affinity of the MC3R antagonist with other melanocortin receptors (e.g., MC1R, MC2R, MC4R, MC5R). In some embodiments, an MC3R antagonist binds MC3R selectively over MC4R. In some embodiments, an MC3R antagonist binds MC3R with an affinity that is at least 2-fold greater (e.g., 2×, 3×, 4×, 5×, 6×, 7×, 8×, 9×, 10×, 20×, 30×, 40×, 50×, 60×, 70×, 80×, 90×, 100×, or more) than the binding affinity of the MC3R antagonist with MC4R.

In some embodiments, an MC3R antagonist inhibits the activity of MC3R selectively over other melanocortin receptors (e.g., MC1R, MC2R, MC4R, MC5R). In some embodiments, an MC3R antagonist inhibits the activity of MC3R at least 2-fold greater (e.g., 2×, 3×, 4×, 5×, 6×, 7×, 8×, 9×, 10×, 20×, 30×, 40×, 50×, 60×, 70×, 80×, 90×, 100×, or more) than other melanocortin receptors (e.g., MC1R, MC2R, MC4R, MC5R). In some embodiments, an MC3R antagonist inhibits the activity of MC3R selectively over MC4R. In some embodiments, an MC3R antagonist inhibits the activity of MC3R at least 2-fold greater (e.g., 2×, 3×, 4×, 5×, 6×, 7×, 8×, 9×, 10×, 20×, 30×, 40×, 50×, 60×, 70×, 80×, 90×, 100×, or more) than MC4R.

In some embodiments, an MC3R antagonist is co-administered with an additional agent or therapy. In some embodiments, the co-administered agent is for the treatment or prevention of the same condition/disease/symptom as the MC3R antagonist (e.g., obesity, overeating, etc.). In some embodiments, the co-administered agent is for the treatment or prevention of a side-effect of the MC3R antagonist (e.g., anxiety). In some embodiments, the co-administered agent is for the treatment or prevention of a comorbidity not treated of prevented by the MC3R agonist (e.g., hypertension, diabetes, heart disease, etc.).

In some embodiments, an MC3R antagonist is co-administered with a psychotherapy technique such as behavior therapy, cognitive therapy, psychodynamic therapy, psychoanalytic therapy, group therapy, family counseling, art therapy, music therapy, vocational therapy, humanistic therapy, existential therapy, transpersonal therapy, client-centered therapy (also called person-centered therapy), Gestalt therapy, biofeedback therapy, rational emotive behavioral therapy, reality therapy, response based therapy, Sandplay therapy, status dynamics therapy, hypnosis and validation therapy. Any suitable psychotherapy techniques, including those listed above, may be co-administered with an MC3R antagonist for the treatment/prevention of appropriate conditions/diseases (e.g., obesity, overeating, etc.), drug side-effects, and/or comorbidities. In some embodiments, an MC3R antagonist is co-administered with a weight loss medication, or a weight loss program involving diet and/or exercise. Suitable weight loss medications for co-administration may include , but are not limited to orlistat (Xenical), lorcaserin (Belviq), phentermine-topiramate (Qsymia), naltrexone-bupropion (Contrave), liraglutide (Saxenda), phentermine, benzphetamine, diethylpropion, phendimetrazine, etc. In some embodiments, am MC3R antagonist is co-administered with a drug that acts upon the GLP1 receptor, such as liraglutide, dulaglutide, exenatide, exenatide extended release, semaglutide, lixisenatide, etc. In some embodiments, an MC3R antagonist is co-administered with liraglutide. In some embodiments, an MC3R antagonist is co-administered with a weight loss drug, such as Contrave (Naltrexone Hydrochloride and Bupropion Hydrochloride), Qysmia (Phentermine and Topiramate), and Belviq (lorcaserin hydrochloride), etc.

In some embodiments, an MC3R antagonist is co-administered with an antianxiety agent. Suitable antianxiety medications for co-administration may include selective serotonin reuptake inhibitors, serotonin-norepinephrine reuptake inhibitors, tricyclics, benzodiazepines (e.g., alprazolam (Xanax), chlordiazepoxide (Librium), diazepam (Valium), lorazepam (Ativan) etc.), beta-blockers (e.g., atenolol (Tenormin), propranolol (Inderal), etc.), buspirone (BuSpar), monoamine oxidase inhibitors, etc.

In some embodiments, an MC3R antagonist is co-administered with a mood stabilizer. Suitable mood stabilizers for co-administration may include lithium, anticonvulsants (e.g., valproate, lamotrigine, carbamazepine, etc.), etc.

In some embodiments, an MC3R antagonist is co-administered with a stimulant. Suitable stimulants for co-administration may include amphetamine/dextroamphetamine (Adderall), dextroamphetamine (Dexedrine, ProCentra, Zenzedi), dexmethylphenidate (Focalin), methylphenidate (Ritalin), amphetamine sulfate (Evekeo), methylphenidate (Ritalin SR, Metadate ER, Methylin ER), amphetamine (Adzenys XR-ODT, Dyanavel XR), dexmethylphenidate (Focalin XR), dextroamphetamine (Adderall XR), lisdexamfetamine (Vyvanse), methylphenidate (Concerta, Daytrana, Jornay PM, Metadate CD, Quillivant XR, Quillichew ER, Ritalin LA), etc.

In some embodiments, an MC3R antagonist is co-administered with any agent or medication suitable for the treatment of the eating disorders and/or mental illnesses described herein.

### III. Administration

In some embodiments, any suitable routes and/or modes of administering the agents (e.g., MC3R agonist, MC3R antagonist/inhibitors, co-administered agent, etc.) find use in embodiments herein. In some embodiments, the compositions and methods described herein act upon the central nervous system (CNS) and therefore routes and/or modes of administration that facilitate entry of the agents into the CNS are utilized. In some embodiments, the compositions and methods described herein act upon the brain of a subject and therefore routes and/or modes of administration that facilitate entry of the agents into the brain (e.g., allow agents to cross the blood-brain barrier) are utilized. In some embodiments, the compositions and methods described herein act upon the hypothalamus of a subject and therefore routes and/or modes of administration that facilitate delivery of the agents to the hypothalamus are utilized. In some embodiments, the compositions and methods described herein act upon the arcuate nucleus of the hypothalamus of a subject and therefore routes and/or modes of administration that facilitate delivery of the agents to the arcuate nucleus are utilized. In some embodiments, the compositions and methods described herein act upon the AgRP neurons of a subject and therefore routes and/or modes of administration that facilitate delivery of the agents to AgRP neurons are utilized. In some embodiments, the compositions and methods described herein act upon the POMC neurons of a subject and therefore routes and/or modes of administration that facilitate delivery of the agents to POMC neurons are utilized.

In some embodiments, routes of administration, formation of the desired agent, and the pharmaceutical composition are selected to provide efficient and effective delivery. In some embodiments, the therapeutic agents herein (e.g., MC3R agonist, MC3R antagonist/inhibitors, co-administered agent, etc.) are provided in pharmaceutical formulations for administration to a subject by a suitable route. The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, the pharmaceutical compositions described herein (e.g., comprising an MC3R agonist, an MC3R antagonist/inhibitors, a co-administered agent, etc.) are formulated into any suitable dosage form, including but not limited to, aqueous oral dispersions, liquids, gels, syrups, elixirs, slurries, suspensions, aerosols, fast melt formulations, effervescent formulations, lyophilized formulations, tablets, powders, pills, dragees, and capsules.

One may administer the compounds and/or compositions in a local rather than systemic manner, for example, via injection of the compound directly into an organ or tissue, often in a depot preparation or sustained release formulation. Such long acting formulations may be administered by implantation (for example subcutaneously or intramuscularly) or by intramuscular injection. Furthermore, one may administer the drug in a targeted drug delivery system, for example, in a liposome coated with organ-specific antibody. The liposomes will be targeted to and taken up selectively by the organ. In addition, the drug may be provided in the form of a rapid release formulation, in the form of an extended release formulation, or in the form of an intermediate release formulation.

Pharmaceutical preparations for oral use can be obtained by mixing one or more solid excipients with the therapeutic agent (e.g., an MC3R agonist, an MC3R antagonist/inhibitors, a co-administered agent, etc.) with any suitable substituents and functional groups disclosed herein, optionally grinding the resulting mixture, and processing the mixture of granules, after adding suitable auxiliaries, if desired, to obtain tablets, pills, or capsules. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

In some embodiments, agents are delivered by inhalation. For administration by inhalation, the agents described herein (e.g., an MC3R agonist, an MC3R antagonist/inhibitors, a co-administered agent, etc.) may be in a form as an aerosol, a mist or a powder. In some embodiments, pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Buccal formulations that include the agents described herein (e.g., an MC3R agonist, an MC3R antagonist/inhibitors, a co-administered agent, etc.) may be administered using a variety of formulations which include, but are not limited to, U.S. Pat. Nos. 4,229,447, 4,596,795, 4,755,386, and 5,739,136.

In some embodiments, the agents described herein (e.g., an MC3R agonist, an MC3R antagonist/inhibitors, a co-administered agent, etc.) are delivered transdermally. Transdermal formulations described herein may be administered using a variety of devices including but not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144; incorporated by reference in their entireties.

In some embodiments, the agents described herein (e.g., an MC3R agonist, an MC3R antagonist/inhibitors, a co-administered agent, etc.) are delivered by parenteral administration (e.g., intramuscular, subcutaneous, intravenous, epidural, intracerebral, intracereroventricular, etc.). Formulations suitable for parenteral administration may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Agents described herein (e.g., an MC3R agonist, an MC3R antagonist/inhibitors, a co-administered agent, etc.) may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally recognized in the field. For other parenteral injections, appropriate formulations may include aqueous or nonaqueous solutions, preferably with physiologically compatible buffers or excipients. Such excipients are generally recognized in the field.

In certain embodiments, delivery systems for pharmaceutical agents (e.g., an MC3R agonist, an MC3R antagonist/inhibitors, a co-administered agent, etc.) may be employed, such as, for example, liposomes and emulsions. In certain embodiments, compositions provided herein also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, an agent (e.g., an MC3R agonist, an MC3R antagonist/inhibitors, a co-administered agent, etc.) is administered in a therapeutically effective amount. Thus, a therapeutically effective amount is an amount that is capable of at least partially preventing or reversing a disease, disorder, or symptoms thereof. The dose required to obtain an effective amount may vary depending on the agent, formulation, disease or disorder, and individual to whom the agent is administered.

Determination of effective amounts may involve in vitro assays in which varying doses of agent are administered to cells in culture and the concentration of agent effective for ameliorating some or all symptoms is determined in order to calculate the concentration required in vivo. Effective amounts may also be based in in vivo animal studies. Pharmaceutical compositions may be in unit dosage forms suitable for single administration of precise dosages. In unit dosage form, the formulation is divided into unit doses containing appropriate quantities of one or more agents (e.g., an MC3R agonist, an MC3R antagonist/inhibitors, a co-administered agent, etc.).

Dosing and administration regimes are tailored by the clinician, or others skilled in the pharmacological arts, based upon well-known pharmacological and therapeutic considerations including, but not limited to, the desired level of therapeutic effect, and the practical level of therapeutic effect obtainable.

In some embodiments, and upon the clinician's discretion, the administration of the compounds may be administered for an extended period of time, including throughout the duration of the patient's life in order to treat the disorder or ameliorate or otherwise control or limit the symptoms of the patient's disease.

In a case wherein the patient's status does improve, upon the clinician's discretion the administration of the agents (e.g., an MC3R agonist, an MC3R antagonist/inhibitors, a co-administered agent, etc.) may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days.

The dose reduction during a drug holiday may be from about 10% to about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, once improvement of the patient's symptoms/disorder/condition has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

In some embodiments, the amount of a given agent that will correspond to such an amount will vary depending upon factors such as the particular compound, disease and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.02-about 5000 mg per day, in some embodiments, about 1-about 1500 mg per day. The desired dose may conveniently be presented in a single dose or as divided doses administered simultaneously (or over a short period of time) or at appropriate intervals, for example as two, three, four or more sub-doses per day.

As discussed above, provided in certain embodiments herein are combination therapies in which an MC3R agonist or an MC3R antagonist/inhibitor is co-administered with an additional agent for the treatment of the disorder/condition, a side effect of the primary agent, or a comorbidity of the disorder/condition. Co-administered agents do not have to be administered in the same pharmaceutical composition, and may, because of different physical and chemical characteristics, have to be administered by different routes. Co-administered agents may be administered concurrently (in the same or separate formulations/compositions) or at separate times (separated by minutes, hours, days, etc.) The co-administered agents may be administered concurrently (e.g., simultaneously, essentially simultaneously or within the same treatment protocol) or sequentially, depending upon the nature of the disease, disorder, or condition, the condition of the patient, and the actual choice of agent used. The determination of the order of administration, and the number of repetitions of administration of each therapeutic agent during a treatment protocol, is well within the knowledge of the clinician after evaluation of the disease being treated and the condition of the patient.

Therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For combination therapies described herein, dosages of the co-administered agents will of course vary depending on the type of co-drug employed, on the specific drug employed, on the disease being treated and so forth. In addition, when co-administered with one or more biologically active agents, the compound provided herein may be administered either simultaneously with the biologically active agent(s), or sequentially.

### EXPERIMETAL

### Example 1

### Melanocortin 3 Receptor Engages AgRP Circuitry to Regulate Feeding and Anxiety

### Materials and Methods

### Animals

Experiments were performed on adult (8-16 weeks old) male and female mice. Mice were group housed on a 12-h light/12-h dark cycle and provided ad libitum access to food and water prior to stereotaxic surgeries or behavioral experiments. Mice were group housed for behavioral experiments (open field test, elevated plus maze, novelty suppressed feeding test) with the exception of cannulated mice, which were single caged for at least one week prior to behavioral experiments. For feeding assays mice were single housed for at least one week before starting feeding measurements. Mouse strains used in this study included C57/BL6J (Jackson Labs), AgRP-ires-Cre (Jackson Labs), MC3R-KO (bred in house),

MC3R loxTB/TB (Jackson Labs), and MC3R-Cre. MC3R-Cre mice were created by the Vanderbilt Transgenic Mouse Resource (Ref. 18; herein incorporated by reference in its entirety). Mice were bred and maintained on a C57/BL6J background except for AgRP-ires-Cre mice which were maintained on a mixed background.

### Viral Vectors

Adeno associated viral vectors used in this study included AAVS-Ef1a-DIO-hM3Dq-mCherry and AAV2-Ef1a-DIO-hM4Di-mCherry. Viral vectors were purchased from Addgene

### Stereotaxic Viral Injections and Cannula Placement

Stereotaxic viral injections were performed as previously described (Refs. 18, 55-57; herein incorporated by reference in their entireties). Mice were anesthetized with isoflorene and placed in a stereotaxic frame (Kopf). A micro-precision drill was used to drill a small burr-hole directly above the viral injection or cannula insertion point and dura was removed. AAV viral vectors were injected into the arcuate nucleus using a micromanipulator (Narishige) attached to a pulled glass pipette. For chemogenetic activation experiments virus was unilaterally injected while bi-lateral injections were performed for chemogenetic inhibition. Viral injection coordinates for the arcuate nucleus were as follows: A/P: −1.5 mm (from bregma), M/L: 0.2 mm, D/V: 5.8 mm (from surface of brain). AgRP-Cre mice were injected in the arcuate nucleus coordinates described above with 250 nl of virus. Virus was injected at a rate of 50 nl/minute over five minutes. Following viral injection, the glass pipette was left in place for an additional five minutes to prevent leaking of virus outside the targeted brain region. To target cre-dependent viral vectors to the arcuate nucleus of MC3R-cre mice 30 nl of virus was injected at a rate of 5 nl/minute over five minutes.

For pharmacological assays an injection cannula was inserted into the lateral ventricle. Cannulas were inserted at the following coordinates: A/P: −0.4 mm, M/L 1 0 mm, D/V: 2.0 mm Cannula's were attached to the scull using Metabond. Two weeks was allotted post-surgery to allow for viral expression and the mice to recover from surgical procedures.

### Pharmacology

Clozapine-N-Oxide (CNO) was purchased from Enzo Life Sciences. On the day of experiments fresh stock CNO (1 mg/0.1 ml) was prepared in water. CNO was administered via i.p. injections (200 ul Saline; 1 mg/kg). Synthesis and in vitro characterization of FMMC-5, PG990 and PG992 were previously described³¹⁻³³. FMMC-5 and PG990 were administered via i.p. injection (10 mg/kg, 200 ul, 10% DMSO). PG992 was administered i.c.v. (5 ug; 500 nl DMSO).

### Feeding Behavior Assays

Behavioral assays were performed on ad libitum fed mice. Mice were single caged for at least one week prior to starting feeding assays. Feeding assays were performed during the light period.

### MC3R Agonist Feeding Assays

For peripheral administration of MC3R agonist compounds, mice were first habituated to vehicle injections (i.p.; saline in 10% DMSO; 200 ul) for 2-4 consecutive days. Following habituation and consecutive days of stable food intake in response to vehicle injections, agonist compounds were administered (i.p.; 10 mg/kg; saline in 10% DMSO). Food intake was measured following drug or vehicle administration by carefully removing food from the food hopper and weighing on a scale. Cages were changed daily during acute feeding assays to prevent spillage of food from the food hopper.

For central administration of MC3R agonist compounds we first administered vehicle injections (500 nl DMSO) for 2-4 consecutive days to habituate mice to handling and injection. Following consecutive days of stable food intake in response to vehicle, we administered the MC3R agonist PG992 (5 ug, 500 nl DMSO). Food intake was measured and ascending time-points following vehicle or PG992 injections. Body weight was measured daily immediately prior to injections.

### DREADD Feeding Assays

To evaluate the effects of DREADD mediated manipulation of ARC-MC3R neurons on feeding and body weight, mice were habituated to twice daily vehicle injections (i.p., saline; 200 ul). Following habituation, the DREADD agonist CNO was administered (i.p., 1 mg/kg, 200 ul) twice daily. Injections were performed at 10 am and 6 pm. Food intake was measured twice daily (10 am and 6 pm) during chronic i.p. injection experiments. Body weight was measured daily at 10 am immediately prior to administering CNO/saline.

### Social Isolation Induced Anorexia

To perform social isolation induced anorexia experiments, daily body weight and food intake was recorded from group housed mice (2-5 mice per cage). Food intake was calculated by dividing the daily food intake by the number of mice in each cage. Following 2-5 days of daily food intake and body weight measurements, mice were individually housed. Acute food intake was recorded at 1 hour, 2 hours, and 4 hours post single housing as well as daily food intake and body weight.

### Restraint Stress Induced Anorexia

Restraint stress induced anorexia experiments were performed on group housed mice (2-5 mice per cage). Prior to beginning restraint, daily food intake and body weight was recorded for five days (baseline). Food and water were removed from each cage for thirty minutes daily from 12 pm-1230 pm to account for the time that mice are without food and water during restraint stress. Following baseline measurements, mice were restrained daily for thirty minutes in 50 ml conical tubes from 12 pm-1230 pm. Daily food intake and body weight was measured at 12 pm. Following 5 days of restraint, food intake and body weight was measured in the same fashion as the baseline period to test for rebound effects following restraint.

### Anxiety Behavior Assays

Anxiety behavioral assays were performed in the following order with 2-4 days separated between each anxiety assay: elevated plus maze, open field test, novelty suppressed feeding test. Behavioral assays were performed during the light cycle between 10 am-5 pm. Open field and elevated plus maze testing was performed in ad libitum fed mice with one exception. Chemogenetic inhibition assays (elevated plus maze and open field test) were performed on mice fasted overnight (6pm-10am) to evaluate inhibition of ARC-MC3R neurons during a time when these cells are expected to be most active (following a fast).

### Open Field Test

Open field tests were performed as previously described (Refs. 55-57; herein incorporated by reference in their entireties). Mice were placed in the corner of a 50 cm×50 cm open field chamber. The center of the arena was marked as the 25 cm×25 cm area in the center of the open field. Exploratory activity was recorded for five minutes using ANYmaze software (Stoelting). Entries to the center, time in the center, distance in the center, and total distance traveled were automatically recorded with video tracking software. For PG992 experiments, mice were randomly assigned to receive either vehicle or PG992 injections. One week later the order of treatments was reversed such that each mouse received both vehicle and PG992 treatment in random order. For DREADD mediated assays, mice were administered CNO on the day of testing. Differences in open field activity between DREADD transduced mice and WT littermates were compared on testing day in response to CNO treatment. Similarly, MC3R KO and WT mice were testing in the open field on the same day in random order and differences in exploratory activity were calculated as previously described.

### Elevated Plus Maze

To perform elevated plus maze tests mice were initially placed into the closed arm of the elevated plus maze arena. The arena consisted of 2 50 cm long arms. One arm was enclosed by walls while the other arm was open. Activity in the maze was recorded for five minutes using animal tracking software (ANYmaze, Stoelting). Latency to enter the open arms, total distance traveled, entries to open arms, time in open arms, and distance traveled in the open arms was automatically calculated using tracking software. Treatment schedules for the elevated plus maze were identical to those described for the open field test.

### Novelty Suppressed Feeding Test

Prior to testing, mice were fasted overnight (6 pm-10 am). NSF testing was performed in the open field arena described above. Briefly, a single pellet of food (3-4 grams) was placed in the center of the open field. Mice were placed in the corner of the open field and their activity was automatically scored for ten minutes using animal tracking software. Latency to eat the food was manually recorded during testing. Feeding was scored as five consecutive seconds of chewing on the food.

### Immunofluorescence and Imaging

To measure changes in neuronal activity in response to CNO, florescent immunohistochemistry was performed to detect cfos. Mice were transcranially perfused with 1×PBS followed by 10% formalin. Following perfusion, brains were fixed for an additional 24 hours in 10% formalin. Brains were then switched to a 20% sucrose solution (in 1×PBS) until the brains sank in the solution (1-3 days) and which point 35 um thick hypothalamic sections were obtained using a cryostat (Leica). Sections were first incubated for 1 hour in blocking buffer (1×PBS with 2% BSA and 0.1% tween-20). Primary antibody (rabbit anti-cfos, Millipore) diluted in blocking buffer was added to sections overnight. After three washes in 1×PBS, secondary antibody was added for 2 hours (donkey anti-rabbit 488). After three additional wash steps sections were mounted on slides and imaged on a confocal microscope (Nikon A1).

### Statistical Analysis

Following DREADD viral injections and behavioral experiments, mice were perfused and location of viral injections were verified via florescence imaging. Animals with no viral expression or no expression in the arcuate nucleus were excluded from analysis. Data were analyzed with Graphpad software.

### Results

### Sexual Dimorphism of the Melanocortin-3 Receptor Circuitry

A striking feature of AN is the extreme sexual dimorphism, with 90-95% of cases in women, and the characteristic age of onset following puberty. In a cohort we collected and characterized with Dr. Julie O'Toole at the Kartini Clinic (Table 1), for example, we observed an average age of onset of approximately 14 years. Thus, while a neuropsychiatric disorder, restricting-type AN may involve a sexually dimorphic neurodevelopmental etiology.

TABLE 1
Phenotypic data of AN probands from the Katrini Clinic (n = 24-35)
AN propositus

| | | History of | Other | |
|---|---|---|---|---|
| | | obsessive | anxiety | |
| Gender | Perfectionism | compulsive traits | disorders | Age of Onset |
| Female 21/24 | 9/24 37% | 9/24 37% | 8/24 33% | 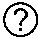 |
| 87% | | | | |

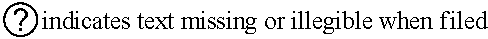

The neural circuitry defined by expression of the MC3R is at the nexus of reproductive and feeding circuits (FIG. 15). Examining postmortem transcriptomics data, a profound sexual dimorphism of human MC3R expression was also observed, with approximately 2-3× more MC3R expression in males vs females, in contrast to the MC4R, found in equal amounts in most brain regions (FIG. 16A). This finding was recapitulated in mice using a variety of methods (FIG. 16B-G). Furthermore, the effects of the MC3R on the effect of reproductive hormones on feeding behavior and energy homeostasis are profound. For example, it has been demonstrated that the hyperphagia seen in the middle trimester of pregnancy in the mouse is highly dependent on the MC3R.

### MC3R is Widely Expressed in a Variety of Arcuate Neurons

MC3R is abundantly expressed in the arcuate nucleus (refs. 16-17; herein incorporated by reference in their entireties). However, the relative abundance of MC3R in specific arcuate cell populations, such as the anorexic POMC and vGLUT2 neurons and the orexigenic AgRP neurons has been unknown (Refs. 8, 21; herein incorporated by reference in their entireties). Given that these arcuate cell populations exert opposing effects on feeding, experiments were conducted during development of embodiments herein to determine if MC3R is enriched in orexigenic or anorexic cell types in the arcuate nucleus. To quantitatively define the cell types in the arcuate containing MC3R RNAscope florescence in situ hybridization was used to quantify MC3R expression in the orexigeneic AgRP neurons and the anorexic POMC neurons. MC3R is diffusely expressed throughout the arcuate nucleus (FIG. 1). Receptor expression is observed in both AgRP and POMC neurons, in addition to other un-identified cell types. MC3R is expressed at a much greater frequency and abundance in orexigenic AgRP neurons, relative to anorexigenic POMC neurons. AgRP neurons contained a significantly greater number of MC3R mrna transcripts than POMC neurons.

### Melanocortin-3 Receptor Activation Recapitulates Activation of AgRP Neurons

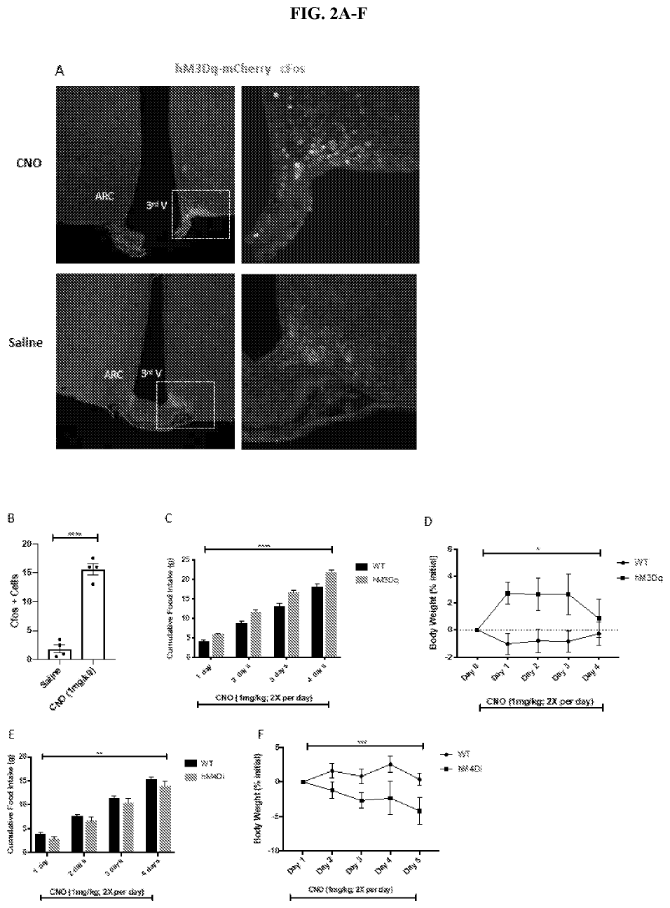

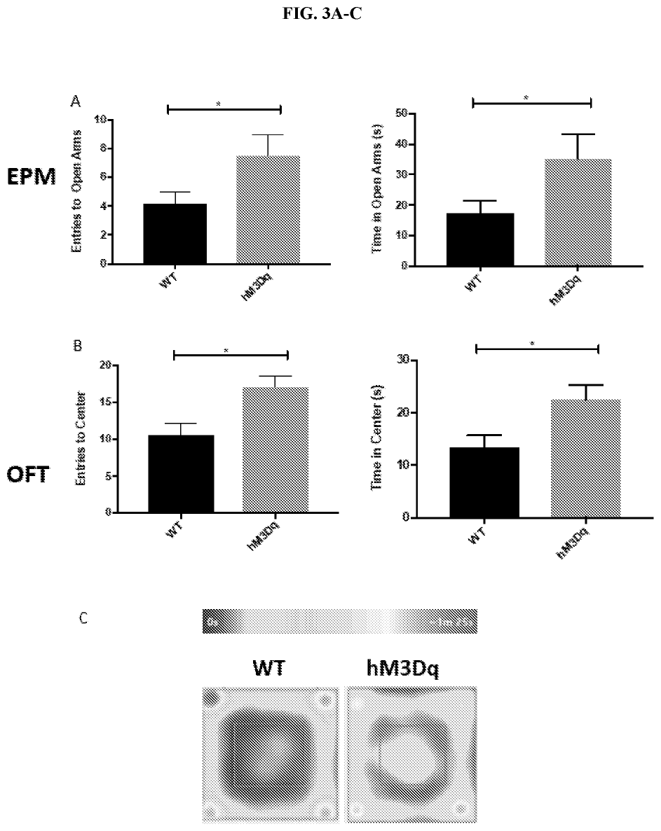

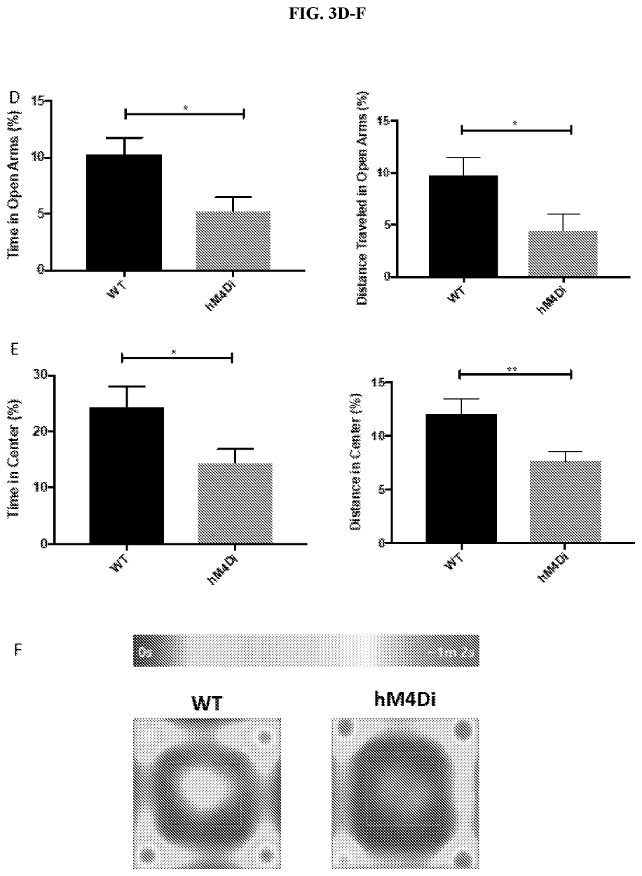

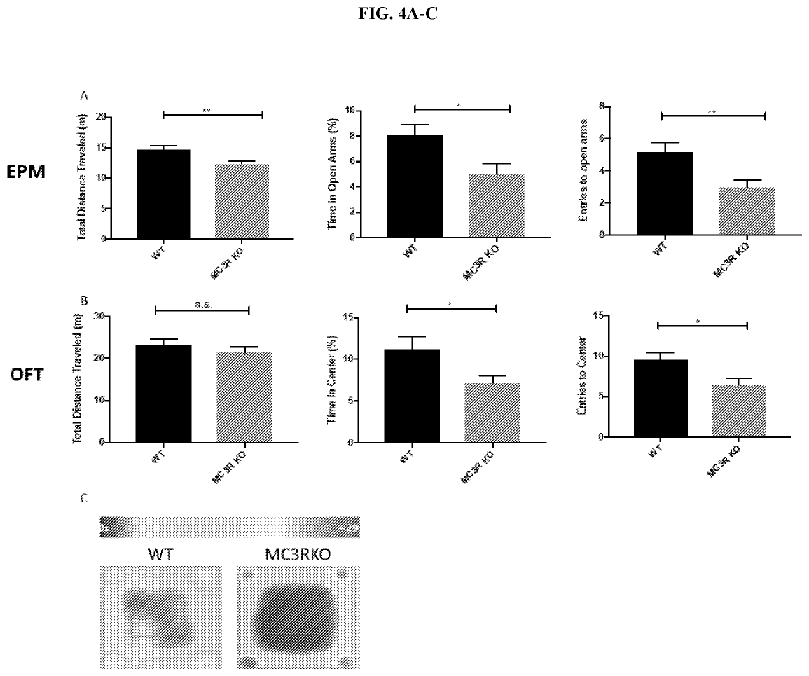

Optogenetic activation of arcuate AgRP neurons has been demonstrated to potently stimulate food intake, even in the face of competing physiological and motivational states, including satiety, anxiety, and fear. Thus, experiments were conducted during development of embodiments herein to determine if DREADD activation of arcuate MC3R neurons would phenocopy this novel property of AgRP neuron activation. Indeed, activation of MC3R ARC neurons produced profound hyperphagia and weight gain, while inhibition had the opposite effect (FIG. 2). In the novelty-suppressed feeding test assay, mice are fasted, and then given access to food in the center of an open field, to test the competition between the desire to feed versus the anxiety of retrieving food from a new location in the center of an open field. Comparable to optogenetic activation of AgRP neurons, DREADD activation of ARC MC3R neurons in WT mice reduced anxiety elevated plus maze and open field tests, while inhibition of ARC MC3R neurons increased anxiety; data shows male and female mice, and no sex differences were noted (FIG. 3). We proceeded to evaluate male and female MC3RKO mice in the novelty suppressed feeding assay, and observed a profound sexual dimorphism (FIG. 17). While knockout of the MC3R had no effect in male knockout vs WT mice, female MC3RKO mice exhibited extreme anxiety, failed to enter the center of the open field to retrieve food, and failed to eat when eating was linked with the anxiogenic stimulus of food placement in the center of an open field (FIG. 17).

### Arcuate MC3R Neurons can Bidirectionally Regulate Feeding and Weight Gain

Given the enhanced expression of MC3R in the orexigenic arcuate AgRP neurons, relative to POMC and vGLUT2 neurons, experiments were conducted during development of embodiments herein to determine whether the dominant role of MC3R in the arcuate is to stimulate food intake and increase body weight. A previously characterized (Ref. 18; herein incorporated by reference in its entirety) MC3R-Cre mouse model in which Cre recombinase is exclusively expressed in cells containing MC3R was utilized for these experiments. To selectively activate the MC3R expressing neurons in the arcuate, a Cre dependent AAV viral vector expressing the DREADD (designer receptor exclusively activated by designer drugs) activator hM3Dq was stereotaxically targeted to the arcuate nucleus in MC3R-Cre mice (Refs 22-23; herein incorporated by reference in their entireties) (FIG. 2). It was validated that CNO administration activates ARC-MC3R neurons by measuring the neuronal activity marker, cfos, in response to CNO or saline injections. Dense cfos expression was observed in ARC-MC3R neurons following CNO injections, indicating that our DREADD approach efficiency activates ARC-MC3R neurons (FIG. 2). To determine if chronic activation of ARC-MC3R neurons is a viable strategy for increasing food intake and body weight CNO was administered twice daily for five consecutive days. Long term activation of ARC-MC3R neurons increased food intake and body weight (BW) (FIG. 2). Cumulative food intake remained elevated relative to littermate control treated animals at the conclusion of the study. Consistently, body weight was significantly increased following one day of treatment and remained elevated at the end of five days of CNO treatment (FIG. 2). No change in food intake or body weight was observed following CNO injections in littermate control mice, indicating that at the dose administered CNO does not exert any effect on food intake or body weight (FIGS. 2, 9). Together, these findings show that despite MC3R expression in both orexigenic and anorexic arcuate neurons, stimulation of arcuate neurons containing MC3R exerts an overall orexigenic effect on feeding.

Activation of ARC-MC3R neurons establishes that these cells are sufficient to increase food intake and body weight and indicates that chronic manipulation of ARC-MC3R cells stimulates feeding and body weight. Experiments were conducted during development of embodiments herein to determine if these neurons are necessary for normal feeding and body weight control by using the inhibitory DREADD hM4Di. In contrast to hM3Dq, peripheral administration of CNO to hM4Di transduced neurons results in both neuronal hyperpolarization and inhibition of presynaptic release, effectively silencing neurons (Refs. 23-24; herein incorporated by reference in their entireties). To selectively inhibit the ARC-MC3R cells a cre-dependent viral vector expressing hM4Di was targeted to the arcuate nucleus in MC3R-Cre mice. CNO induced inhibition of ARC-MC3R neurons reduced both food intake and BW following twice daily administration (FIG. 2). No effect on BW was observed in WT littermate control mice following CNO injections, indicating that the observed effects were not due to adverse effects of CNO (FIG. 2). These results indicate that manipulation of ARC-MC3R neurons bidirectionally regulates both feeding and body weight.

### Arcuate MC3R Neurons can Bidirectionally Regulate Anxiety

Hunger or activation of AgRP neurons reduces anxiety behavior (Refs. 14-15, 25-26; herein incorporated by reference in their entireties). Experiments were conducted during development of embodiments herein to determine if selective activation of ARC-MC3R neurons would phenocopy the reduced anxiety observed following AgRP neuron activation. In the elevated plus maze test of anxiety, acute chemogenetic activation of ARC-MC3R neurons increased entries to the open arms and time in the open arms, indicating reduced anxiety behavior (FIG. 3a-b). Consistently, in the open field test, acute activation of ARC-MC3R neurons increased entries in the center and time in the center (FIG. 3c-d). In another test of anxiety, the novelty suppressed feeding test (NSF), acute stimulation of ARC-MC3R neurons decreased latency to consume food and increased time in the center, also indicating reduced anxiety (FIG. 10). No significant differences in locomotion were detected in any anxiety assays following activation of ARC-MC3R neurons, demonstrating that these effects were not due to elevated locomotion (FIG. 11).

To further determine if ARC-MC3R neurons are necessary for regulating anxiety behavior the chemogenetic inhibitor hM4Di was used to selectively inhibit ARC-MC3R neurons. In the elevated plus maze, inhibition of ARC-MC3R neurons reduced time spent in the open arms and distance traveled in the open arms, indicating increased anxiety behavior (FIG. 3). Consistently, in the open field test, inhibition of ARC-MC3R neurons was associated with reduced time in the center and reduced distance traveled in the center, indicating increased anxiety-like behavior (FIG. 3). No significant differences in locomotion were detected in the EPM or OFT following inhibition of ARC-MC3R neurons (FIG. 11). These findings show that manipulation of ARC-MC3R neurons bidirectionally regulates anxiety behavior.

### Lack of MC3R Results in Elevated Anxiety and Enhanced Stress-Induced Anorexia

Experiments were conducted during development of embodiments herein to determine if subjects lacking MC3R (e.g., MC3R knock-out (KO) mice) display behavioral characteristics resembling anorexia. In the elevated plus maze test, MC3R KO mice entered the open arms less frequently, spent less time in the open arms, and traveled less, indicating increased anxiety (FIG. 4a-c). In the open field test, MC3R KO mice entered the center less frequently and spent less time in the center of the open field (FIG. 4d, e). Total distance traveled was not different in MC3R KO mice in the OFT, indicating that these changes are likely due to elevated anxiety (FIG. 4f). To confirm that lack of MC3R is associated with elevated anxiety, the elevated plus maze test was performed in a second MC3R KO mouse strain (MC3R loxTB/TB). MC3R loxTB/TB mice displayed enhanced anxiety behavior in the elevated plus maze (FIG. 13).

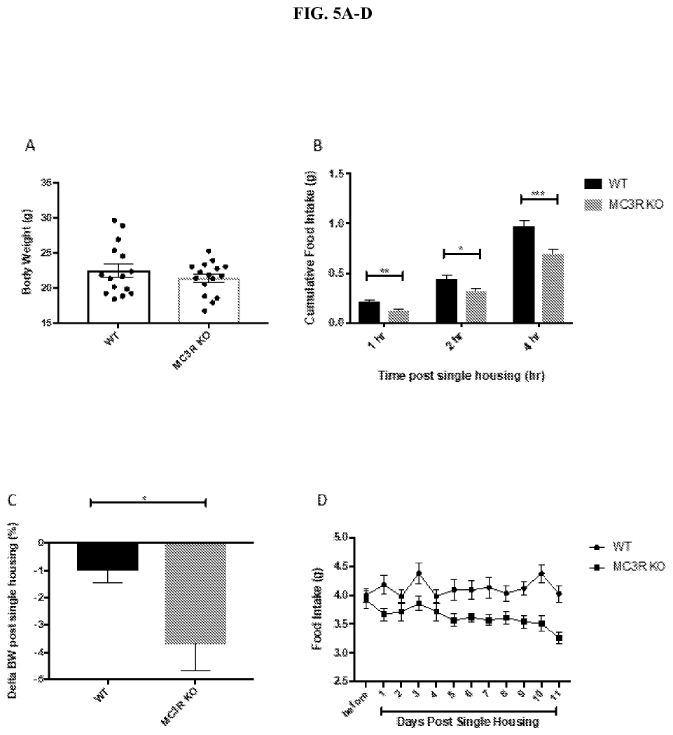

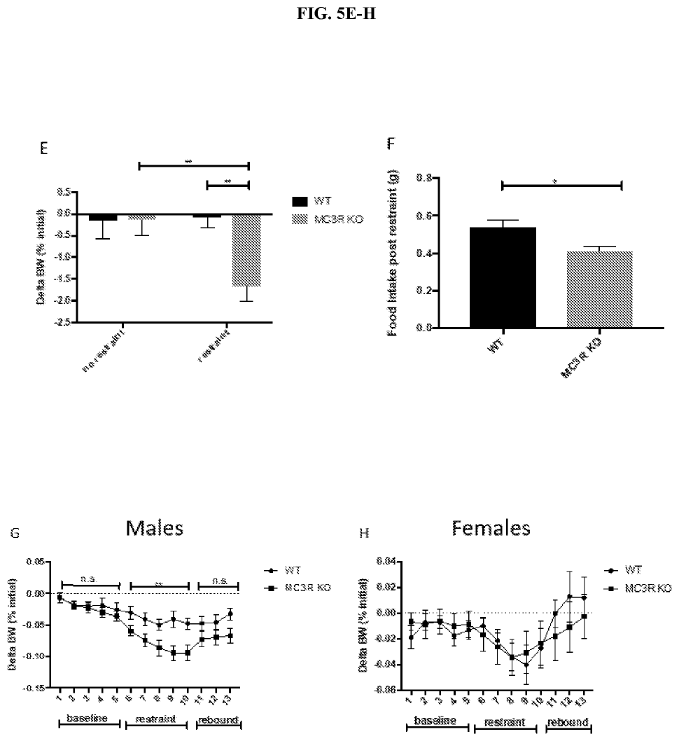

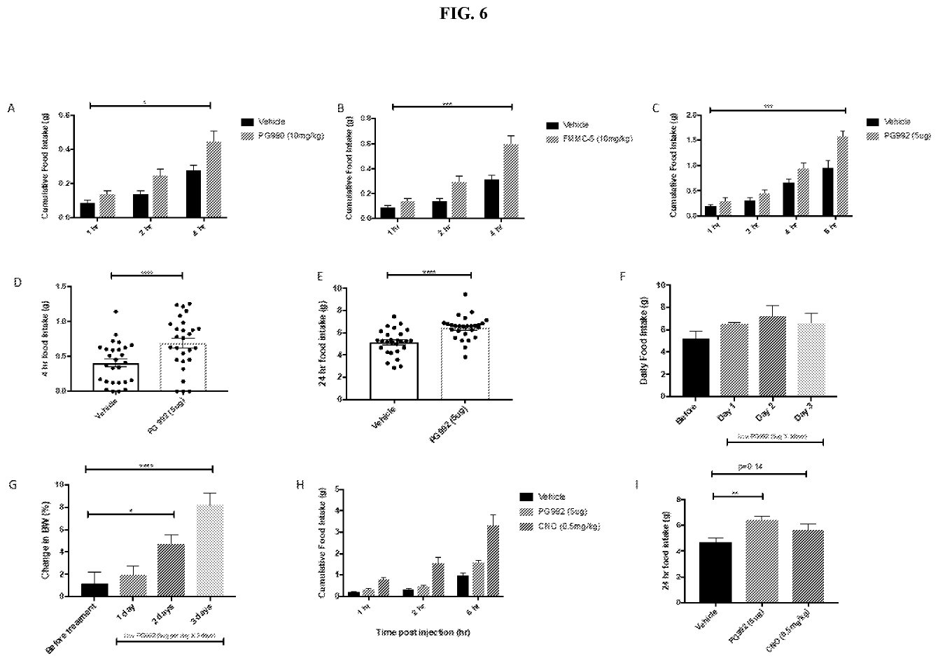

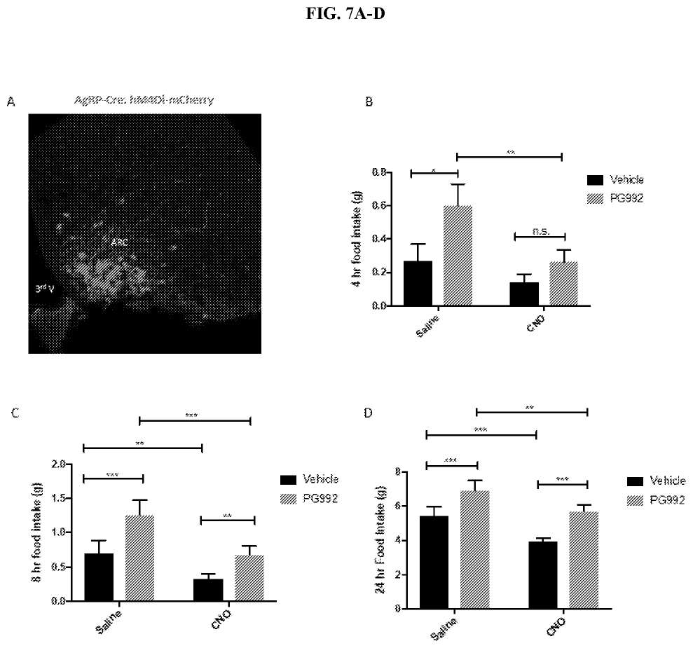

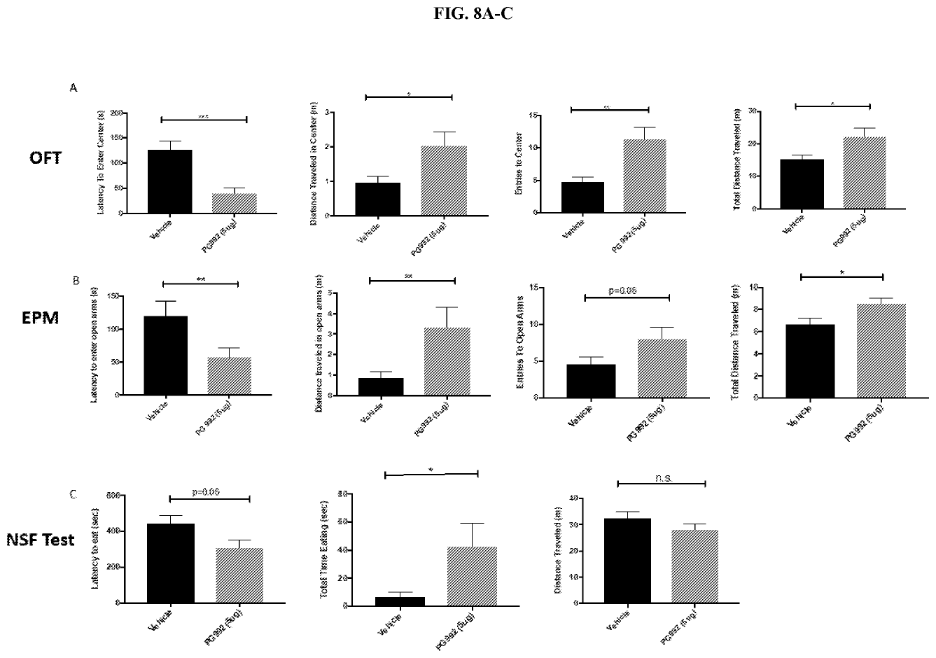

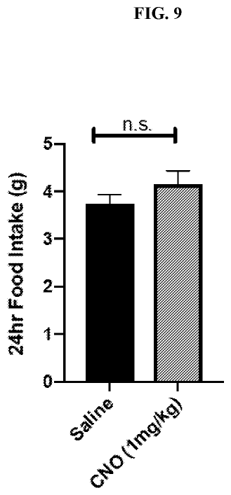

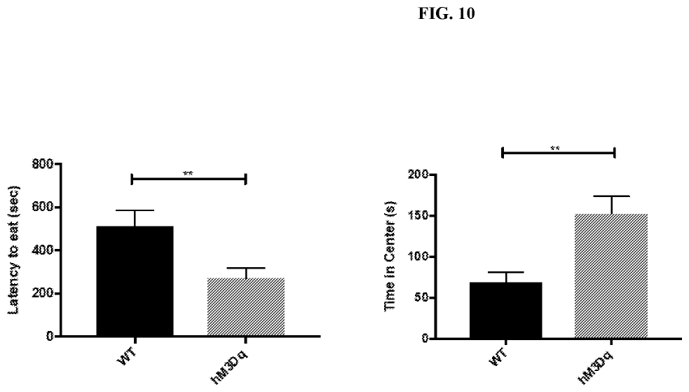

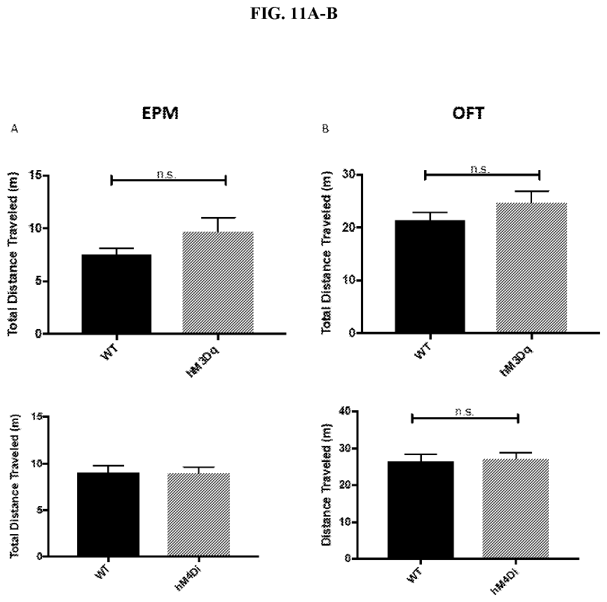

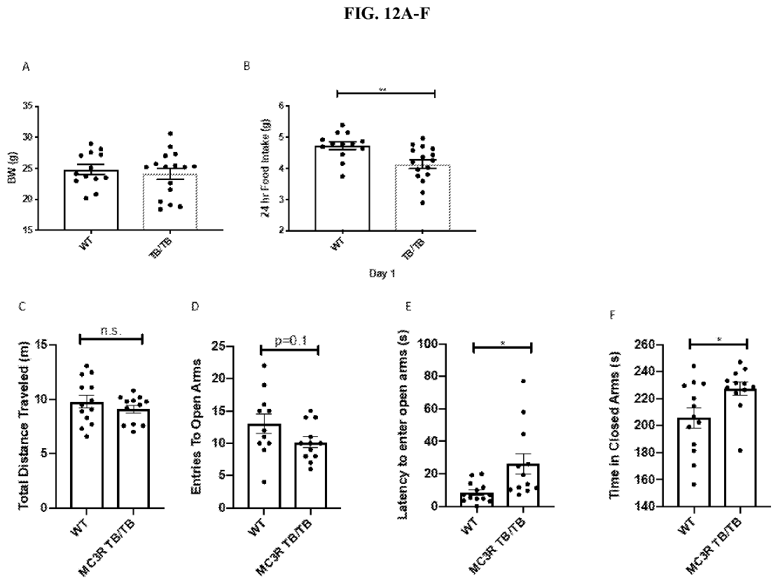

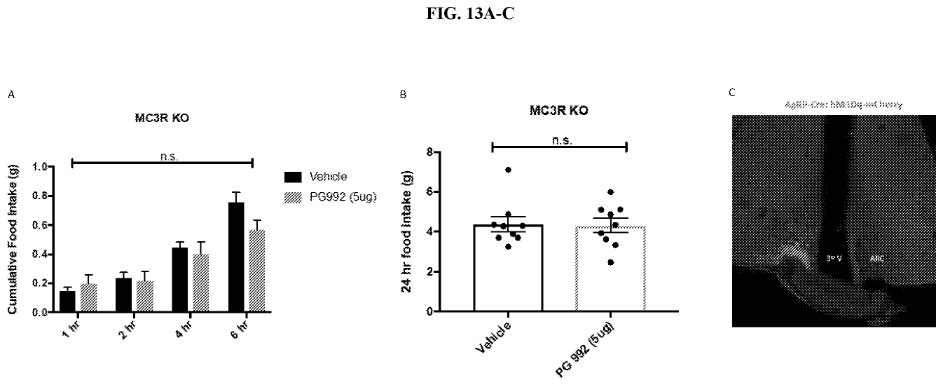

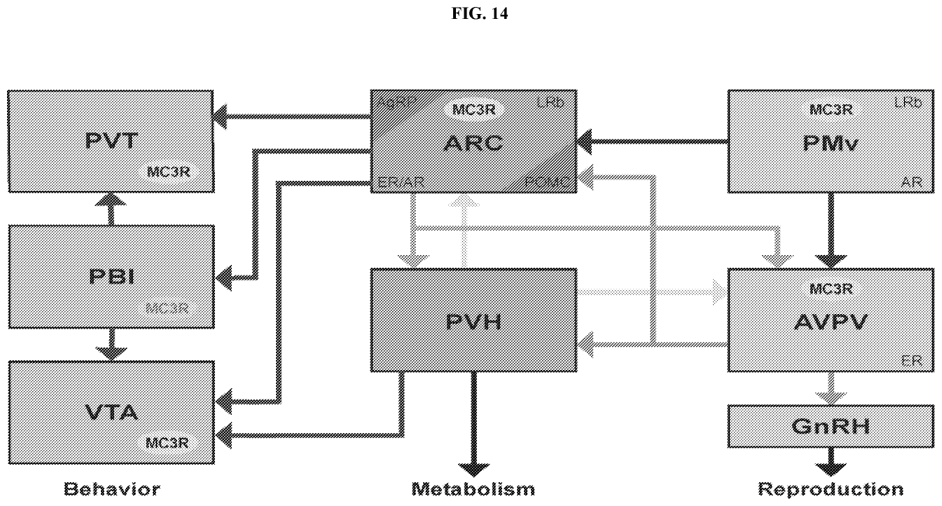

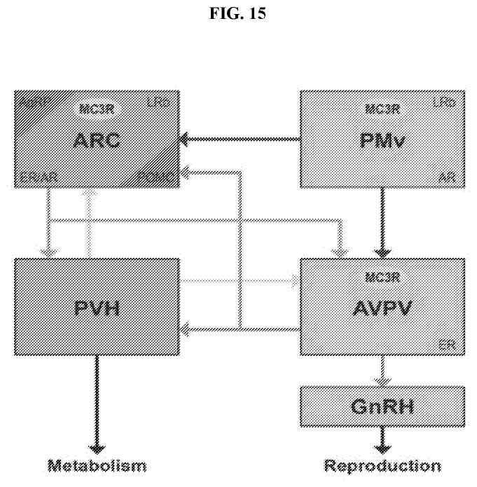

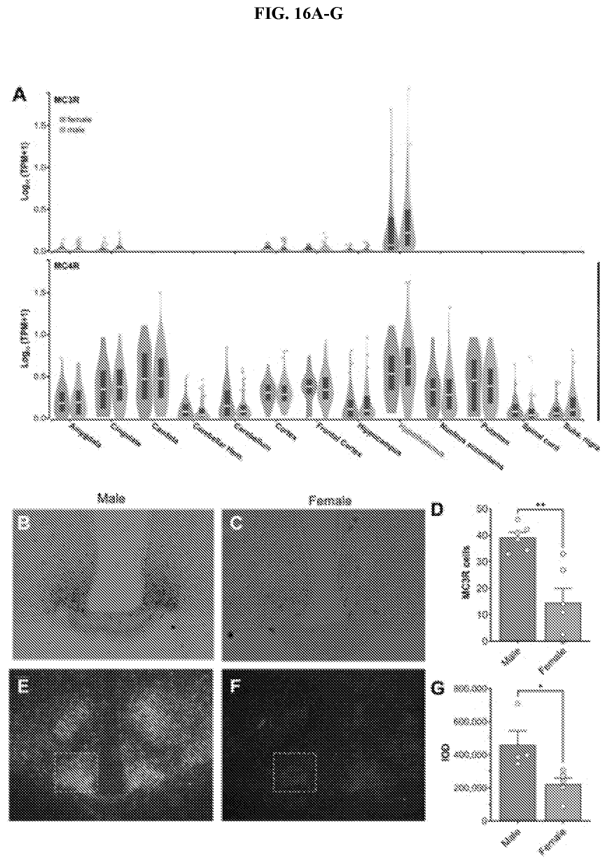

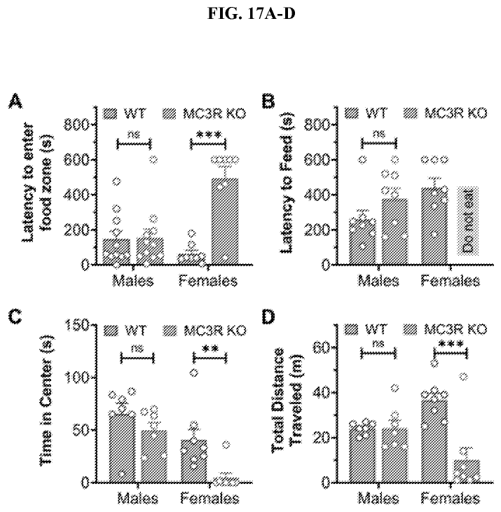

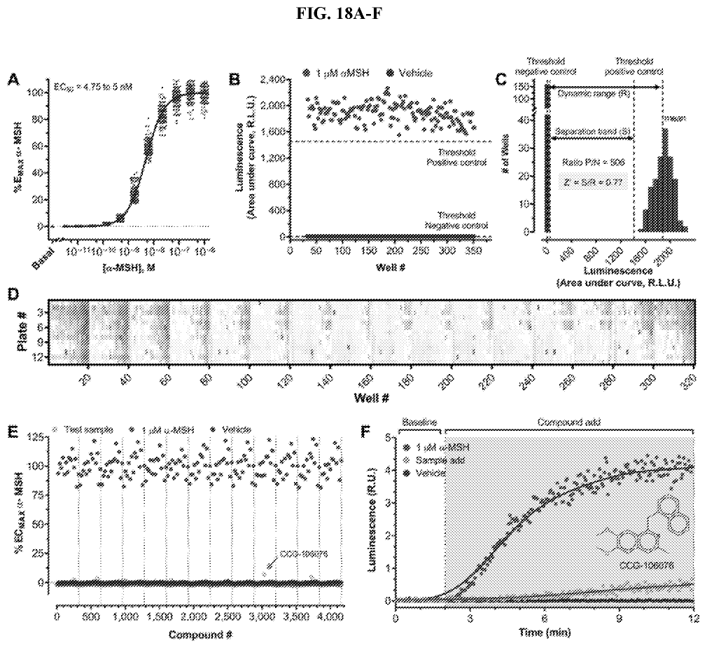

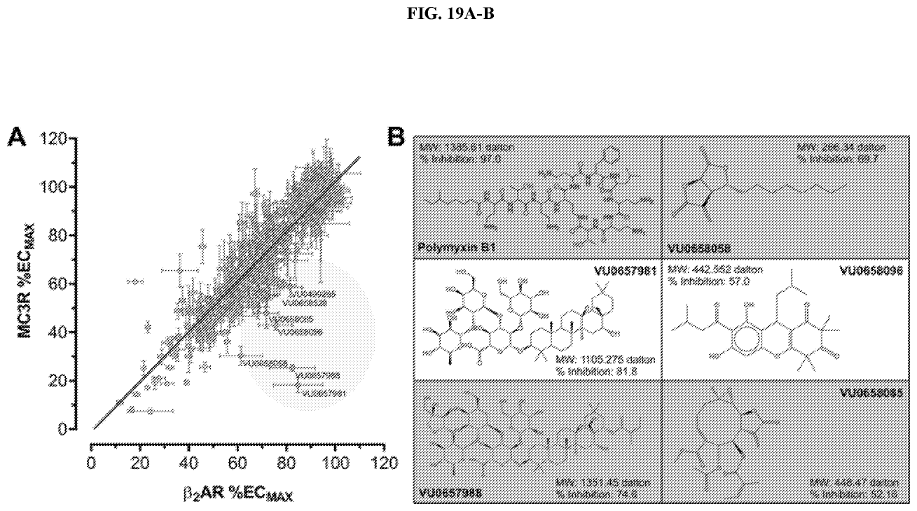

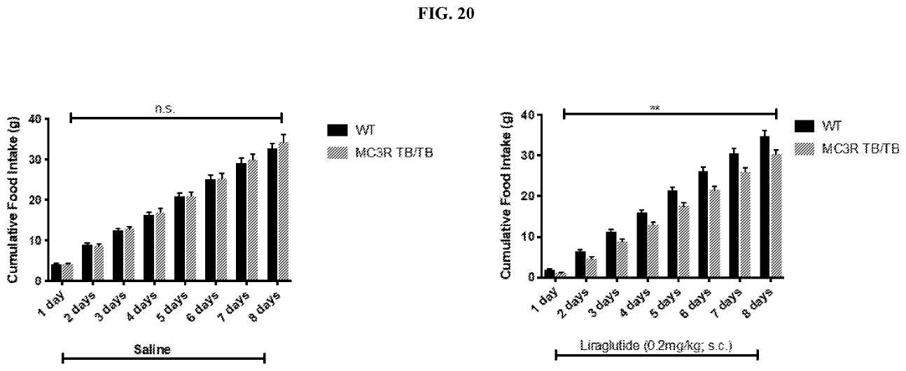

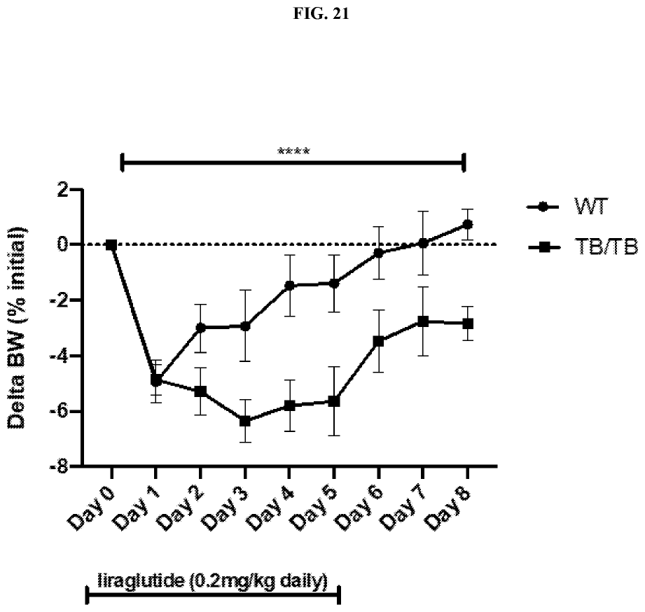

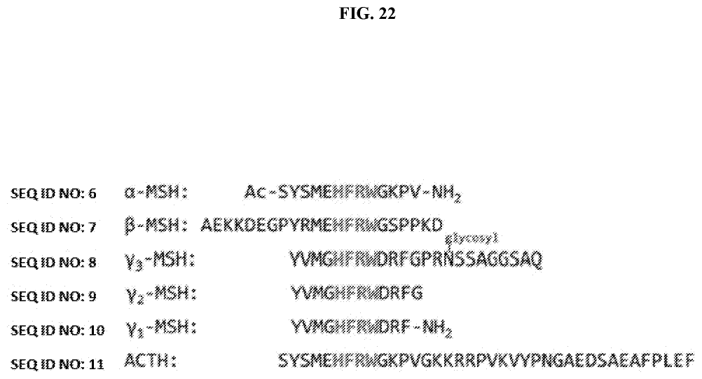

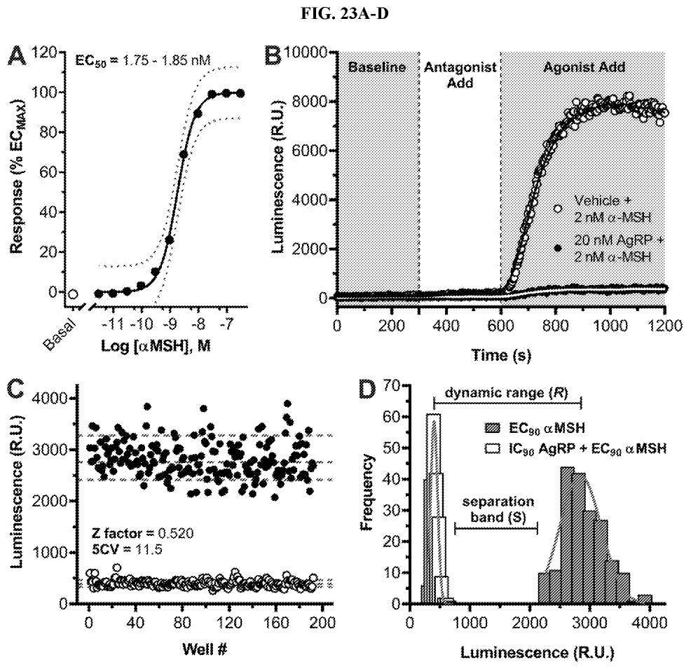

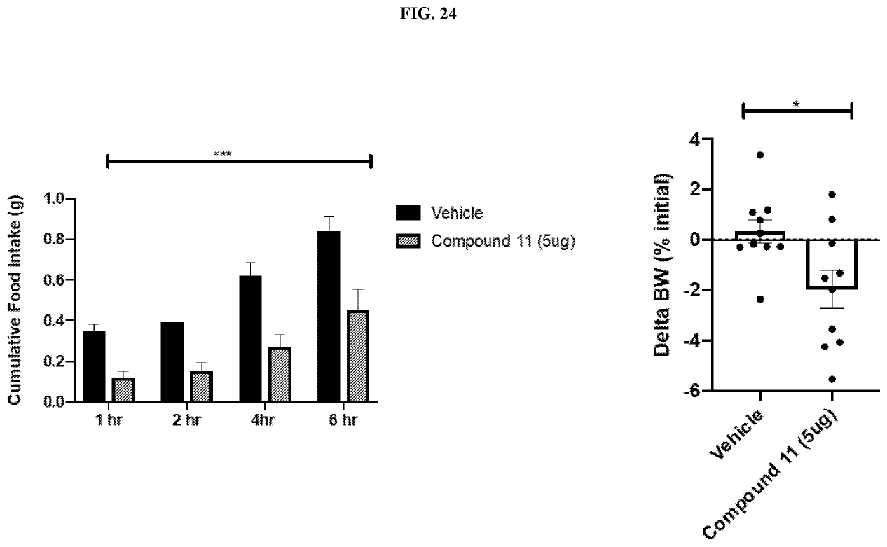

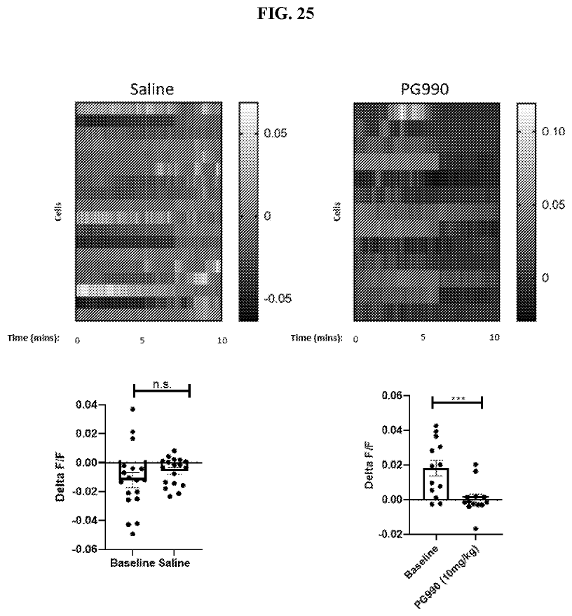

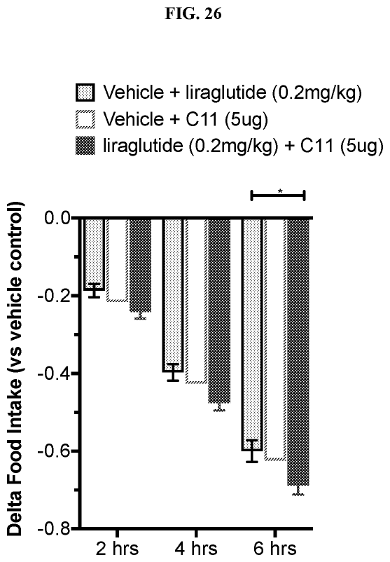

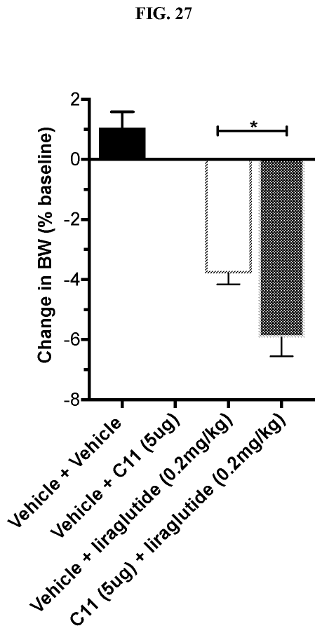

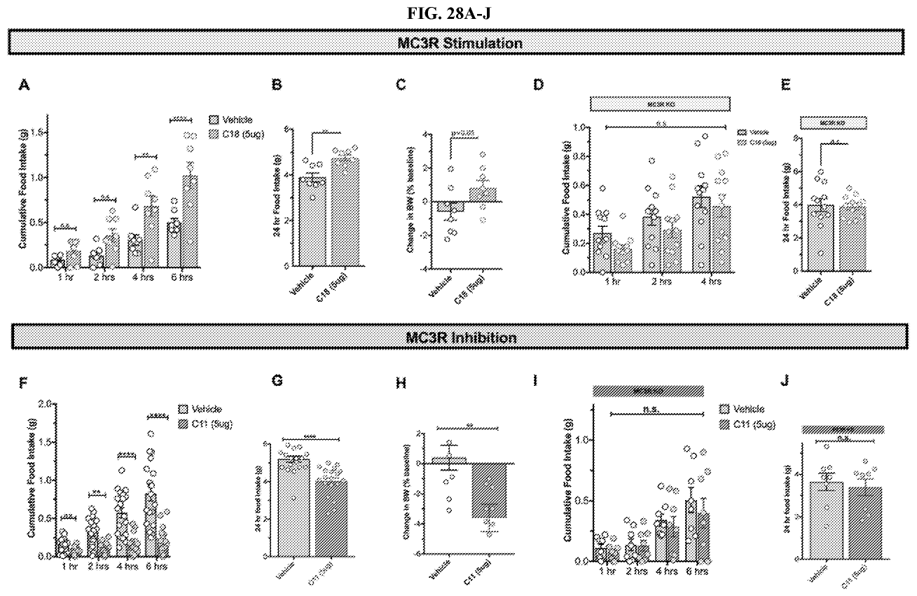

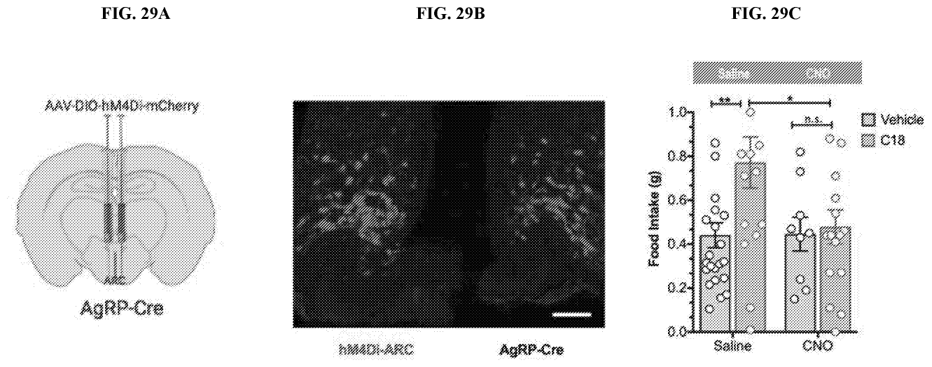

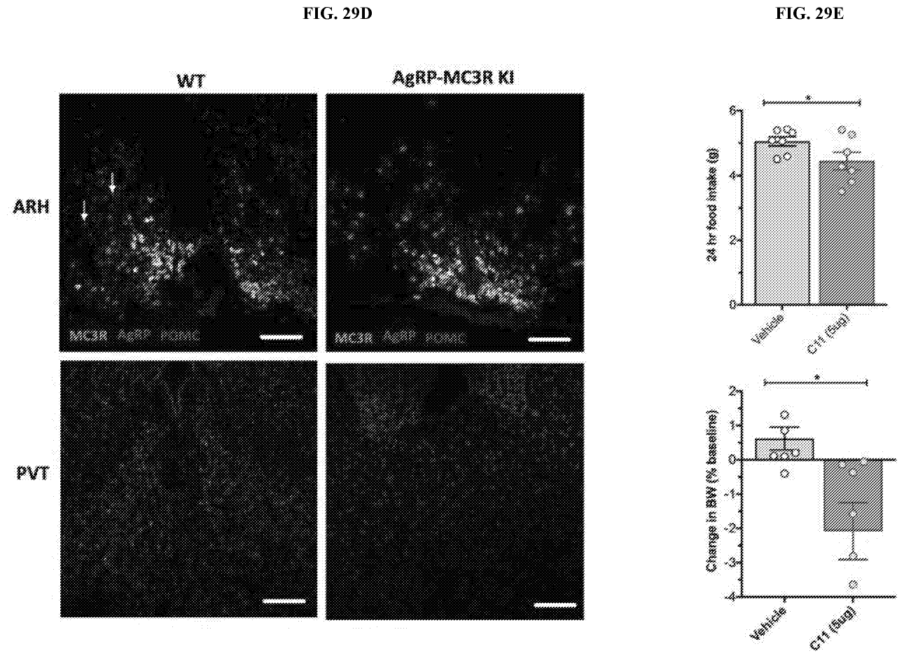

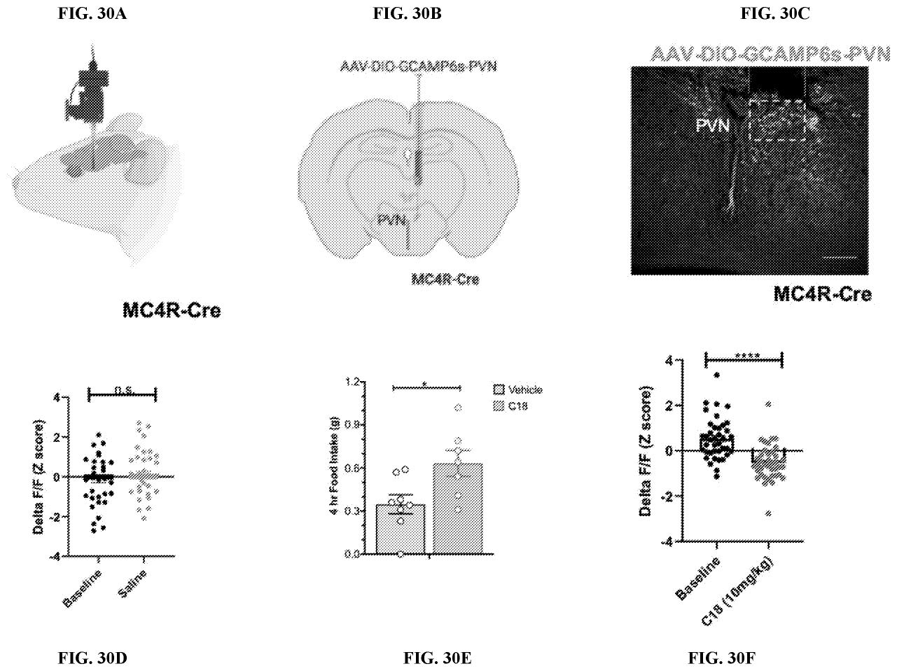

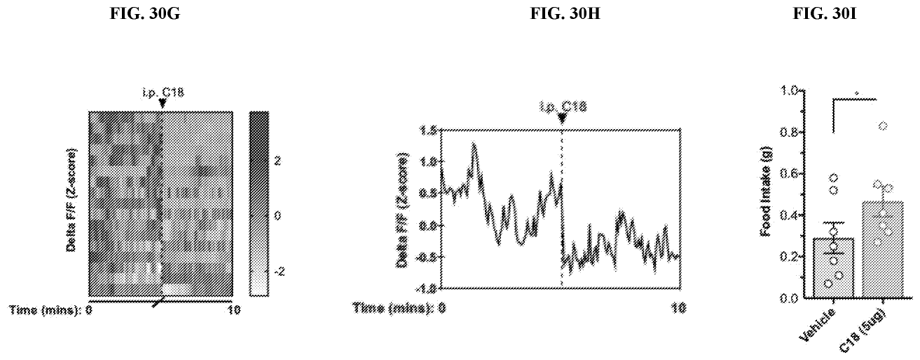

Experiments were conducted during development of embodiments herein to determine whether MC3R is important for regulating food intake and anxiety in response to stressful stimuli. To test the role of MC3R in regulating stress induced anorexia, social isolation induced anorexia experiments were performed. Social isolation has been previously validated to cause profound and long-lasting negative affective behaviors in mice, including suppression of body weight and food intake (Refs. 27-28; herein incorporated by reference in their entireties). No significant difference in food intake or body weight was observed between group housed MC3R KO and WT mice prior to social isolation (FIG. 5d). However, upon single housing MC3R KO mice consumed less food and lost more weight than WT littermate control mice (FIG. 5b-d). Reductions in food intake were long lasting, lasting out to 11 days post single housing (FIG. 5d). Similar enhanced susceptible to social isolation induced anorexia was also observed in MC3R loxTB/TB mice FIG. 13).

As a second assay to measure stress-induced anorexia, restraint-stress induced anorexia experiments were conducted in MC3R KO and littermate control mice (Refs. 29-30; herein incorporated by reference in their entireties). Consistent with single cage anorexia findings, MC3R KO mice consumed less food than WT mice following restraint and lost more body weight than WT littermates following a single acute restraint stress (FIG. 5e, f). Upon testing the effects of continued daily restraint stress on BW in WT and MC3R KO mice, male MC3R KO mice displayed an enhanced stress induced reduction in BW following daily restraint (FIG. 5g). This effect was not observed in female MC3R KO mice, indicating some sex differences in restraint stress assays.

### MC3R-Specific Agonists Potently Stimulate Food Intake and Weight Gain

Experiments were conducted during development of embodiments herein to characterize a number of different MC3R peptide agonists (Table 2) in order to seek a compound with therapeutic potential as an orexigenic agent. PG990 series of peptides (PG990 and PG992) increases 4 hr food intake 2-3× (FIG. 6A), increases 24 h food intake (FIG. 6B), and produce 8% weight gain in just 3 days of treatment (FIG. 6C). This activity is centrally mediated, as no activity on food intake or body weight was observed when PG992 was administered icy to MC3RK0 mice. Remarkably, icy administration of PG992 is even more potent inducer of 24 h food intake than DREADD activation of MC3R ARC neurons (FIG. 6D). Data with the weakest of these compounds, D-Trp8-γ-MSH, shows a trend towards reduction of anxiety in the elevated plus maze assay (FIG. 6E) after a single peripheral injection. ICV administration of PG992 shows a clear anxiolytic activity in three different behavioral assays, the open field test, elevated plus maze, and novelty suppressed feeding assay (FIG. 8). Thus, the data demonstrates that treatment with MC3R agonist phenocopies DREADD activation of MC3R ARC neurons.

TABLE 2
Pharmacological properties of exemplary MC3R peptide agonists.

| | | MC3R | MC3R | MC4R | MC4R | |
|---|---|---|---|---|---|---|
| Compound | | EC₅₀ | IC₅₀ | EC₅₀ | IC₅₀ | |
| Name | Compound Structure | (nM) | (nM) | (Nm) | (Nm) | Ref |
| D-Trp8-γ- | H-Tyr-Val-Met-Gly-His-Phe- | 0.33 | 6.7 | 100 | 600 | 1 |
| MSH | Arg-DTrp-Asp-Arg-Phe-Gly-OH | | | | | |
| PG990 | Ac-Nle-c[Asp-Pro-Pro-DNal(2′)- | 1.9 | 2.4 | >1000 | 190 | 2 |
| | Arg-Trp-Lys]-NH₂ | | | | | |
| PG992 | Ac-Nle-c[Asp-Trp-Pro-DNal(2′)- | 42 | 11 | >1000 | 950 | 2 |
| | Arg-Trp-Lys]-NH₂ | | | | | |
| FMMC-5 | [Ac6c-His-DPhe-Arg-Trp-Asp]- | 30 | 10 | 280 | 260 | 3 |
| | NH₂ | | | | | |

The efficacy was tested of two MC3R specific agonists to regulate food intake following peripheral administration. Both compounds, PG990 and FMMC-5, stimulated food intake by up to 50% following a single peripheral injection to ad libitum fed mice (FIGS. 6a and 6b). These effects lasted between 6-8 hours, with no significant difference observed in 24-hour food intake or body weight. Experiments were conducted during development of embodiments herein to demonstrate to increase the potency of the hyperphagia in response to MC3R agonism by directly administering a highly selective MC3R agonist, PG992, directly into the central nervous system via i.c.v. injection. Consistent with PG990 and FMMC-5 effects on feeding, administration of PG992 (5 μg) potently stimulated food intake by nearly 50% following central injection (FIGS. 6c and 6d). In contrast to peripheral administration, central administration of PG992 stimulated food intake for at least 24 hours following a single injection (FIG. 6e). No effect on food intake was observed in MC3R KO mice, indicating that the observed effects are mediated by MC3R (FIG. 14). Similar findings were seen for another MC3R agonist, compound 18 (FIGS. 28-30).

Consecutive once daily i.c.v. injections of PG992 were administered to test for continued effects on food intake and body weight. Treatment with PG992 increased daily 24-hour food intake on each day of treatment (FIG. 6f), relative to pre-treatment daily food intake. Furthermore, body weight increased by 2-3 percent each treatment day, increasing approximately 8 percent following three days of treatment (FIG. 6g). To compare the hyperphagia induced by pharmacological stimulation of MC3R to AgRP neuron stimulation, a Cre dependent AAV viral vector expressing the DREADD activator hM3Dq was stereotaxically targeted to the arcuate nucleus in AgRP-Cre mice (FIG. 13). Chemogenetic activation of AgRP neurons produced robust hyperphagia (FIGS. 6h and 6i). Acute stimulation of food intake following activation of AgRP neurons was significantly higher than pharmacological stimulation of MC3R (FIG. 6h). However, MC3R activation with PG992 produced a greater change in 24-hour food intake than AgRP neuron activation (FIG. 6i). Taken together, these results indicate that pharmacological stimulation of MC3R potently increases both food intake and BW to comparable levels as AgRP neuron stimulation.

### MC3R-Specific Agonists Stimulate Food Intake via AgRP Neurons

To test if PG992 exerts its effects on food intake via AgRP neurons a chemogenetic inhibition approach was used to selectively inhibit AgRP neurons prior to PG992 administration. To inhibit AgRP neurons, the arcuate nucleus of AgRP-Cre mice was targeted with stereotaxic viral injections of Cre-dependent AAV viral vectors expressing the chemogenetic inhibitor hM4Di (FIG. 7). Central stimulation of MC3R with PG992 robustly stimulated food intake at 4 hours, 8 hours, and 24 hours post PG992 administration (FIG. 7b, c, d). However, prior chemogenetic inhibition of AgRP neurons significantly reduced the ability of PG992 to stimulate feeding, indicating that MC3R-induced hyperphagia depends, at least in part, on AgRP neuronal circuitry (FIG. 7b, c, d). Similar findings were reported for another MC3R agonist, compound 18 (FIG. 29).

### MC3R agonist PG992 Reduces Anxiety in Multiple Behavioral Assays

Disorders of negative energy balance, such as anorexia nervosa and cachexia, are frequently associated with adverse psychological phenotypes, such as increased anxiety (Refs. 34-35; herein incorporated by reference in their entireties). Experiments were conducted during development of embodiments herein to demonstrate that pharmacological stimulation of MC3R modulates anxiety behavior in addition to feeding. Acute i.c.v. injections of PG992 were administered and a series of anxiety behavior assays were performed. In the open field test of anxiety behavior MC3R activation decreased the latency to enter the center, increased the distance traveled in the center, and increased entries to the center (FIG. 8a). PG992 treatment also increased total distance traveled, as has been observed with the appetite stimulant ghrelin (Refs. 36-37; herein incorporated by reference in their entireties) (FIG. 8a). Similar findings were obtained in the elevated plus maze with MC3R stimulation decreasing latency to enter the open arms, while increasing distance traveled in the open arms (FIG. 8b). Entries to the open arms trended towards being higher following PG992 treatment (FIG. 8b). As observed in the open field test, MC3R stimulation also increased total distance traveled in the elevated plus maze (FIG. 8b). In the novelty suppressed feeding test of anxiety behavior, MC3R stimulation decreased latency to feed (FIG. 8c), and increased total time eating (FIG. 8c), without significantly effecting total distance traveled (FIG. 8c). These findings indicate that MC3R stimulation reduces anxiety and increases exploration in an effort to locate and secure food.

### Example 2

### Validation of an HTS Assay for MC3R Agonists

Given the demonstrated Gs-protein subtype coupling for MC3R (Refs. 56-58; incorporated by reference in its entirety), a mammalian cell-based kinetic luminescence-readout assay was developed to screen for small-molecules MC3R orthosteric agonists. A genetically encoded split-firefly luciferase (FLuc) reporter that incorporates the cAMP-binding domain B from protein kinase A regulatory subunit type IIβ (RIIβB) at the luciferase hinge region was ustilized. Binding of cAMP to RIIβB induces an allosteric conformational shift of the linked FLuc N- and C-terminal domains, recreating the catalytic site for D-luciferin oxidation to produce lcAMp-dependent luminescence. A HEK293 clonal cell line expressing cAMP Glo sensor 22F under hygromycin B selection was transfected with the human MC3R (GB Acc. No AY227893) sequence, and a clonal HEK293 cell line (dual hygromycin B and G418 selection) was isolated with a 250 to 300-fold signal-to-noise (maximum response to baseline) ratio when stimulated with a saturating concentration (1 μM) of the endogenous ligand α-MSH (FIG. 18A). These cells enable the measurement of rapid kinetic intracellular cAMP luminescence responses from MC3R activation with α-MSH potencies ranging from 4.75 to 5 nM (EC50 values, range matches 95% confidence interval, 100 data points, 5 independent experiments, FIG. 18A). Baseline signal was minimal, with an ample dynamic range amenable for HTS. Signal window coefficients or Z′-factors65 were determined in three independent experiments performed at three different days per HTS assay validation guidelines published in the National Center for Advancing Translational Sciences Assay Guidance Manual. These validation experiments consisted of determining the cAMP maximum response in the presence of 1 μM (saturating concentration) α-MSH alternating with basal response from vehicle treated cells (FIG. 18B). Typical assay signal window and Z′ factor values are represented with Z′ factor≥0.7, intra-assay percent coefficient of variation (% CV)≤10%, and inter-assay % CV≤20%. These parameters far exceed the recommended threshold for HTS assay inter-assay % CV≤20%. These parameters far exceed the recommended threshold for HTS assay adequacy.

A screen was performed with a series of 4160 samples from the ChemDiv 100,000 compound diversity set maintained at CCG (FIG. 2D-F). Examination of the color-coded intensity assay map (FIG. 18D) demonstrates the occurrence of positional artifacts due to environmental factors such as differential evaporation for a given plate well position and time-drifts between different plates possibly caused by Fluc substrate instability. Other positional biases presented by instrumental liquid-handling errors were also seen. A three-tier approach was adopted to mitigate the effect of these artifacts. First, quality-control plates without compounds and stimulated with a half-maximal (EC₅₀) αMSH concentration (5 nM) were interspersed between the sample plates for a given day. These “positive stimulation” plates were then used to “correct” the responses from the compound assay plates. Alternating positive and negative controls were included (1 μM αMSH and vehicle respectively) in columns 1-2 and 23-24 of each sample plate and used them to normalize assay plates to their individual maximum and minimum response range, and used to compare the responses from different plates as each is normalized to its own response window. A two-dimensional quadratic linear regression model was fitted to the data from the whole campaign that relies on neighboring-well values to adjust the effect of artifacts. FIG. 18E shows a scatterplot of individual sample, and positive and negative control responses for this pilot screen. Efforts to correct for artifact biases were effective in generating an assay window with average Z-factor values equal to 0.69. An activity threshold above three standard deviations (3SD) from negative control average values was used. Six compounds had activities above the threshold (FIG. 18E) ranging from 8.7% of maximum α-MSH effect for the most active sample to 1.5% for the least active. Raw time-course kinetic data (FIG. 18F) for CCG-106076 demonstrates that the uncorrected data is in line with the normalized data from FIG. 18B, demonstrating that the data normalization and bias elimination do not affect the fidelity of raw data.

In the process of validating the HTS assay, a screen for MC3R antagonists was performed on a library of 23,496 compounds from the Vanderbilt chemical library. When hits were counter-screened against the β2AR, all top-ranking hits were purified natural products from the Spectrum collection or NCI's natural product set, with Polymyxin B, an antibiotic isolated from Paenibacillus polymyxa, showing submicromolar potency and four-fold MC3R over MC4R selectivity, despite the fact that >20,000 of the compounds screened were synthetic small molecules (FIG. 19).

### Example 3

### Liraglutide Administration

Liraglutide is a glucagon-like peptide-1 receptor agonist (GLP-1 receptor agonist) also known as incretin mimetics. It works by increasing insulin release from the pancreas and decreases excessive glucagon release. Liraglutide was approved for medical use in Europe in 2009 and in the United States in 2010. Liraglutide has been shown to be effective at inducing and sustaining weight loss in a population of obese patients including those with hypertension, dyslipidemia, type 2 diabetes and obstructive sleep apnea.

Experiments conducted during development of embodiments herein demonstrate that MC3R knockout coupled with Liraglutide administration produced greater inhibition of food intake, greater weight loss, and a lowering of setpoint after liraglutide treatment of the MC3R knockout when compare to Liraglutide administration in the wild type mouse (FIGS. 20-21).

### Example 4

### Antagonist Administration

Based on the data in the MC3RKO mouse, and the MC3R knockdown in the arcuate nucleus, Experiments were conducted during development of embodiments herein to demonstrate MC3R antagonist inhibition of food intake and promotion of weight loss. Using a selective MC3R antagonist (compound 11, ref. 58; incorporated by reference in its entirety), it was demonstrated (FIG. 24) that icy administration of a single dose of 5 ug of cpd 11 into WT C57BL6J mice reduces food intake by nearly 50% (FIG. 24, left panel), and decreases body weight as well (FIG. 24, right panel).

### Example 5

### Co-Administration of MC3R Specific Antagonist and Liraglutide

Experiments were conducted during development of embodiments herein to demonstrate the ability of MC3R specific antagonists to inhibit food intake and reduce body weight as well as to to increase the activity of other weight loss drugs. Compound 11 increased the inhibition of food intake by liraglutide (FIG. 26) and increased the weight loss induced by liraglutide by over 40% (FIG. 27). The data demonstrates the induction of hypophagia and weight loss by each treatment, compared with injection of vehicle. Experiments were conducted in 8-12 week old male C57BL/6J mice.

### REFERENCES

The following references are herein incorporated by reference in their entireties.

1. Andermann, M. L. & Lowell, B. B. Toward a Wiring Diagram Understanding of Appetite Control. Neuron (2017). doi:10.1016/j.neuron.2017.06.014

2. Sternson, S. M., Nicholas Betley, J. & Cao, Z. F. H. Neural circuits and motivational processes for hunger. Current Opinion in Neurobiology (2013). doi:10.1016/j.conb.2013.04.006

3. Sohn, J. W., Elmquist, J. K. & Williams, K. W. Neuronal circuits that regulate feeding behavior and metabolism. Trends in Neurosciences (2013). doi:10.1016/j.tins.2013.05.003

4. Mercer, A. J., Hentges, S. T., Meshul, C. K. & Low, M. J. Unraveling the central proopiomelanocortin neural circuits. Front. Neurosci. (2013). doi:10.3389/fnins.2013.00019

5. Cone, R. D. Anatomy and regulation of the central melanocortin system. Nature Neuroscience (2005). doi:10.1038/nn1455

6. Sternson, S. M. & Atasoy, D. Agouti-related protein neuron circuits that regulate appetite. Neuroendocrinology (2014). doi:10.1159/000369072

7. Krashes, M. J., Shah, B. P., Koda, S. & Lowell, B. B. Rapid versus delayed stimulation of feeding by the endogenously released agRP neuron mediators GABA, NPY, and AgRP. Cell Metab. (2013). doi:10.1016/j.cmet.2013.09.009

8. Zhan, C. et al. Acute and Long-Term Suppression of Feeding Behavior by POMC Neurons in the Brainstem and Hypothalamus, Respectively. J. Neurosci. (2013). doi:10.1523/jneurosci.2742-12.2013

9. Betley, J. N., Cao, Z. F. H., Ritola, K. D. & Sternson, S. M. Parallel, redundant circuit organization for homeostatic control of feeding behavior. Cell (2013). doi:10.1016/j .cell.2013.11.002

10. Atasoy, D., Nicholas Betley, J., Su, H. H. & Sternson, S. M. Deconstruction of a neural circuit for hunger. Nature (2012). doi:10.1038/nature11270

11. Luquet, S., Perez, F. A., Hnasko, T. S. & Palmiter, R. D. NPY/AgRP neurons are essentials for feeding in adult mice but can be ablated in neonates. Science (80-.). (2005). doi:10.1126/science.1115524

12. Krashes, M. J. et al. Rapid, reversible activation of AgRP neurons drives feeding behavior in mice. J. Clin. Invest. (2011). doi:10.1172/JCI46229

13. Aponte, Y., Atasoy, D. & Sternson, S. M. AGRP neurons are sufficient to orchestrate feeding behavior rapidly and without training. Nat. Neurosci. (2011). doi:10.1038/nn.2739

14. Burnett, C. J. et al. Hunger-Driven Motivational State Competition. Neuron (2016). doi:10.1016/j.neuron.2016.08.032

15. Padilla, S. L. et al. Agouti-related peptide neural circuits mediate adaptive behaviors in the starved state. Nat. Neurosci. (2016). doi:10.1038/nn.4274

16. Bagnol, D. et al. Anatomy of an endogenous antagonist: relationship between Agouti-related protein and proopiomelanocortin in brain. J. Neurosci. (1999).

17. Roselli-Rehfuss, L. et al. Identification of a receptor for gamma melanotropin and other proopiomelanocortin peptides in the hypothalamus and limbic system. Proc. Natl. Acad. Sci. (2006). doi:10.1073/pnas.90.19.8856

18. Ghamari-Langroudi, M. et al. Regulation of energy rheostasis by the melanocortin-3 receptor. Sci. Adv. (2018). doi:10.1126/sciadv.aat0866

19. Sutton, G. M. et al. The Melanocortin-3 Receptor Is Required for Entrainment to Meal Intake. J. Neurosci. (2008). doi:10.1523/JNEUROSCI.3615-08.2008

20. Renquist, B. J. et al. Melanocortin-3 receptor regulates the normal fasting response. Proc. Natl. Acad. Sci. (2012). doi:10.1073/pnas.1201994109

21. Fenselau, H. et al. A rapidly acting glutamatergic ARC→*PVH satiety circuit postsynaptically regulated by α-MSH. Nat. Neurosci. (2017). doi:10.1038/nn.4442

22. Armbruster, B. N., Li, X., Pausch, M. H., Herlitze, S. & Roth, B. L. Evolving the lock to fit the key to create a family of G protein-coupled receptors potently activated by an inert ligand. Proc. Natl. Acad. Sci. (2007). doi:10.1073/pnas.0700293104

23. Alexander, G. M. et al. Remote Control of Neuronal Activity in Transgenic Mice Expressing Evolved G Protein-Coupled Receptors. Neuron (2009). doi:10.1016/j.neuron.2009.06.014

24. Stachniak, T. J., Ghosh, A. & Sternson, S. M. Chemogenetic Synaptic Silencing of Neural Circuits Localizes a Hypothalamus→*Midbrain Pathway for Feeding Behavior. Neuron (2014). doi:10.1016/j.neuron.2014.04.008

25. Li, C. et al. AGRP neurons modulate fasting-induced anxiolytic effects. Transl. Psychiatry (2019). doi:10.1038/s41398-019-0438-1

26. Dietrich, M. O., Zimmer, M. R., Bober, J. & Horvath, T. L. Hypothalamic Agrp neurons drive stereotypic behaviors beyond feeding. Cell (2015). doi:10.1016/j.ce11.2015.02.024

27. Zelikowsky, M. et al. The Neuropeptide Tact Controls a Distributed Brain State Induced by Chronic Social Isolation Stress. Cell (2018). doi:10.1016/j.ce11.2018.03.037

28. Ieraci, A., Mallei, A. & Popoli, M. Social Isolation Stress Induces Anxious-Depressive-Like Behavior and Alterations of Neuroplasticity-Related Genes in Adult Male Mice. Neural Plast. (2016). doi:10.1155/2016/6212983

29. Buynitsky, T. & Mostofsky, D. I. Restraint stress in biobehavioral research: Recent developments. Neuroscience and Biobehavioral Reviews (2009). doi:10.1016/j.neubiorev.2009.05.004

30. Campos, A. C., Fogaça, M. V., Aguiar, D. C. & Guimarães, F. S. Animal models of anxiety disorders and stress. Rev. Bras. Psiquiatr. (2013). doi:10.1590/1516-4446-2013-1139

31. Grieco, P., Balse, P. M., Weinberg, D., MacNeil, T. & Hruby, V. J. D-amino acid scan of γ-melanocyte-stimulating hormone: Importance of Trp8 on human MC3 receptor selectivity. J. Med. Chem. (2000). doi:10.1021/jm000211e

32. Merlino, F. et al. Development of Macrocyclic Peptidomimetics Containing Constrained α,α-Dialkylated Amino Acids with Potent and Selective Activity at Human Melanocortin Receptors. J. Med. Chem. (2018). doi:10.1021/acs.jmedchem.8b00488

33. Carotenuto, A. et al. Discovery of Novel Potent and Selective Agonists at the Melanocortin-3 Receptor. J. Med. Chem. (2015). doi:10.1021/acs.jmedchem.5b01285

34. Herpertz-dahlmann, B., Holtkamp, K. & Konrad, K. Eating disorders: Anorexia and bulimia nervosa. Handb. Clin. Neurol. (2012). doi:10.1016/B978-0-444-52002-9.00026-7

35. Smink, F. R. E., Van Hoeken, D. & Hoek, H. W. Epidemiology, course, and outcome of eating disorders. Current Opinion in Psychiatry (2013). doi:10.1097/YCO0b013e328365a24f

36. Jerlhag, E. et al. Ghrelin stimulates locomotor activity and accumbal dopamine-overflow via central cholinergic systems in mice: Implications for its involvement in brain reward. Addict. Biol. (2006). doi:10.1111/j.1369-1600.2006.00002.x

37. Adermark, L. et al. Ghrelin administration into tegmental areas stimulates locomotor activity and increases extracellular concentration of dopamine in the nucleus accumbens. Addict. Biol. (2007). doi:10.1111/j.1369-1600.2006.00041.x

38. Marks, D. L., Hruby, V., Brookhart, G. & Cone, R. D. The regulation of food intake by selective stimulation of the type 3 melanocortin receptor (MC3R). Peptides (2006). doi:10.1016/j.peptides.2005.01.025

39. Lee, M. et al. Effects of selective modulation of the central melanocortin-3-receptor on food intake and hypothalamic POMC expression. Peptides (2008). doi:10.1016/j.peptides.2007.11.005

40. Lippert, R. N., Ellacott, K. L. J. & Cone, R. D. Gender-specific roles for the melanocortin-3 receptor in the regulation of the mesolimbic dopamine system in mice. Endocrinology (2014). doi:10.1210/en.2013-2049

41. Pandit, R. et al. Melanocortin 3 receptor signaling in midbrain dopamine neurons increases the motivation for food reward. Neuropsychopharmacology (2016). doi:10.1038/npp.2016.19

42. Mavrikaki, M. et al. Melanocortin-3 receptors in the limbic system mediate feeding-related motivational responses during weight loss. Mol. Metab. (2016). doi:10.1016/j.molmet.2016.05.002

43. Pei, H. et al. Lateral Hypothalamic Mc3R-Expressing Neurons Modulate Locomotor Activity, Energy Expenditure, and Adiposity in Male Mice. Endocrinology 160, 343-358 (2018).

44. Sternson, S. M. & Eiselt, A.-K. Three Pillars for the Neural Control of Appetite. Annu. Rev. Physiol. (2016). doi:10.1146/annurev-physiol-021115-104948

45. Rossi, M. A. & Stuber, G. D. Overlapping Brain Circuits for Homeostatic and Hedonic Feeding. Cell Metabolism (2018). doi:10.1016/j.cmet.2017.09.021

46. Sweeney, P. & Yang, Y. Neural Circuit Mechanisms Underlying Emotional Regulation of Homeostatic Feeding. Trends in Endocrinology and Metabolism (2017). doi:10.1016/j.tem.2017.02.006

47. Hay, P. J., Touyz, S. & Sud, R. Treatment for severe and enduring anorexia nervosa: A review. Australian and New Zealand Journal of Psychiatry (2012). doi:10.1177/0004867412450469

48. Bulik, C. M. et al. Prevalence, heritability, and prospective risk factors for anorexia nervosa. Arch. Gen. Psychiatry (2006). doi:10.100¹/ₐrchpsyc.63.3.305

49. Keski-Rahkonen, A. et al. Epidemiology and course of anorexia nervosa in the community Am. J. Psychiatry (2007). doi:10.1176/appi.ajp.2007.06081388

50. Marks, D. L., Butler, A. A., Turner, R., Brookhart, G. & Cone, R. D. Differential role of melanocortin receptor subtypes in cachexia. Endocrinology (2003). doi:10.1210/en.2002-221099

51. Fazeli, P. K. et al. Treatment with a ghrelin agonist in outpatient women with anorexia nervosa: A randomized clinical trial. J. Clin. Psychiatry (2018). doi:10.4088/JCP.17m11585

52. Fazeli, P. et al. Short-term treatment with a ghrelin agonist significantly improves gastric emptying in anorexia nervosa. Endocrine reviews. Conference: 98th annual meeting and expo of the endocrine society, ENDO 2016. United states. Conference start: 20160401. Conference end: 20160404 (2016). doi:10.1210/endo-meetings.2016.OABA.2.SUN-606
53. Carlini, V. P. et al. Ghrelin increases anxiety-like behavior and memory retention in rats. Biochem. Biophys. Res. Commun. (2002).

54. Carvajal, P., Carlini, V. P., Schiöth, H. B., de Barioglio, S. R. & Salvatierra, N. A. Central ghrelin increases anxiety in the Open Field test and impairs retention memory in a passive avoidance task in neonatal chicks. Neurobiol. Learn. Mem. (2009). doi:10.1016/j .nlm.2008.12.008

55. Sweeney, P. & Yang, Y. An excitatory ventral hippocampus to lateral septum circuit that suppresses feeding. Nat. Commun. (2015). doi:10.1038/ncomms10188

56. Sweeney, P. & Yang, Y. An Inhibitory Septum to Lateral Hypothalamus Circuit That Suppresses Feeding. J. Neurosci. (2016). doi:10.1523/jneurosci.2042-16.2016

57. Sweeney, P., Li, C. & Yang, Y. Appetite suppressive role of medial septal glutamatergic neurons. Proc. Natl. Acad. Sci. (2017). doi:10.1073/pnas.1707228114

58. Singh, A., Dirain, M., Witek, R., Rocca J. R., Edison, A. S., and Haskell-Luevano, C. Structure—Activity Relationships of Peptides Incorporating a Bioactive Reverse-Turn Heterocycle at the Melanocortin Receptors: Identification of a 5800-fold Mouse Melanocortin-3 Receptor (mMC3R) Selective Antagonist/Partial Agonist versus the Mouse Melanocortin-4 Receptor (mMC4R). J. Med. Chem. 56, 2747-2763, 2013.

1. A method of treating an eating disorder comprising administering a melanocortin 3 receptor (MC3R) agonist to a subject suffering from the eating disorder.

2. The method of claim 1, wherein the eating disorder is characterized by under eating.

3. The method of claim 1, wherein the eating disorder is characterized by one or more emotional/mental symptoms.

4. The method of claim 3, wherein the eating disorder is characterized by anxiety and/or depression.

5. The method of claim 1, wherein the eating disorder is anorexia nervosa.

6. The method of claim 1, wherein the eating disorder is anorexia nervosa.

7. The method of claim 1, wherein the MC3R agonist is selective for MC3R over melanocortin 4 receptor (MC4R).

8. The method of claim 1, wherein the MC3R agonist is a peptide.

9. The method of claim 8, wherein the peptide comprises an amino acid sequence of SEQ ID NOS: 1-15.

10. The method of claim 9, wherein the peptide comprises an amino acid sequence of SEQ ID NO: 12, wherein Xaa¹ and Xaa⁴ are selected from Table 3.

11. The method of claim 1, wherein the MC3R agonist is a small molecule.

12. The method of claim 1, wherein the administration is repeated on a recurring basis for a period of at least 1 week.

13. The method of claim 12, wherein the administration is repeated on a daily basis.

14. The method of claim 12, wherein the administration is repeated on a recurring basis for a period of at least 1 month.

15. The method of claim 14, wherein the administration is repeated on a recurring basis for a period of at least 1 year.

16. The method of claim 1, wherein the MC3R agonist is co-administered with nutritional therapy, psychotherapy, nasogastric feeding, antidepressant agents, and/or antipsychotic agents.

17. A method of treating an emotional/mental disorder comprising administering a melanocortin 3 receptor (MC3R) agonist to a subject suffering from the emotional/mental disorder.

18. The method of claim 17, wherein the eating disorder is characterized by anxiety and/or depression.

19. The method of claim 17, wherein the MC3R agonist is selective for MC3R over melanocortin 4 receptor (MC4R).

20. The method of claim 15, wherein the MC3R agonist is a peptide.

21. The method of claim 20, wherein the peptide comprises an amino acid sequence of SEQ ID NOS: 1-15.

22. The method of claim 21, wherein the peptide comprises an amino acid sequence of SEQ ID NO: 12, wherein Xaa¹ and Xaa⁴ are selected from Table 3.

23. The method of claim 17, wherein the MC3R agonist is a small molecule.

24. The method of claim 17, wherein the administration is repeated on a recurring basis for a period of at least 1 week.

25. The method of claim 24, wherein the administration is repeated on a daily basis.

26. The method of claim 24, wherein the administration is repeated on a recurring basis for a period of at least 1 month.

27. The method of claim 26, wherein the administration is repeated on a recurring basis for a period of at least 1 year.

28. The method of claim 17, wherein the MC3R agonist is co-administered with psychotherapy, antianxiety agents, mood stabilizers, stimulants, antidepressant agents, and/or antipsychotic agents.

29. A method of treating an eating disorder comprising co-administering a melanocortin 3 receptor (MC3R) antagonist and an antianxiety agent to a subject suffering from the eating disorder.

30. The method of claim 29, wherein the eating disorder is characterized by over eating.

31. The method of claim 29, wherein the eating disorder is characterized by obesity.

32. A pharmaceutical composition comprising an MC3R antagonist and a weight-loss drug.

33. The pharmaceutical composition of claim 32, wherein the MC3R antagonist and a weight-loss drug are separately formulated.

34. The pharmaceutical composition of claim 32, wherein the MC3R antagonist and a weight-loss drug are in a single formulation.

35. The pharmaceutical composition of claim 32, wherein the weight loss drug is a glucagon-like peptide 1 (GLP-1) receptor agonist.

36. The pharmaceutical composition of claim 35, wherein the GLP-1 receptor agonist is selected from aglutide, dulaglutide, exenatide, exenatide extended release, semaglutide, and lixisenatide.

37. The pharmaceutical composition of claims 32, wherein the weight loss drug is Contrave (Naltrexone Hydrochloride and Bupropion Hydrochloride), Qysmia (Phentermine and Topiramate), or Belviq (lorcaserin hydrochloride).

38. A method of treating obesity and/or inducing weight loss comprising administering a pharmaceutical composition of one of claims 32-38.

39. A method of treating obesity and/or inducing weight loss comprising co-administering an MC3R antagonist and a weight-loss drug.

40. The method of claim 39, wherein the weight loss drug is a glucagon-like peptide 1 (GLP-1) receptor agonist.

41. The method of claim 40, wherein the GLP-1 receptor agonist is selected from aglutide, dulaglutide, exenatide, exenatide extended release, semaglutide, and lixisenatide.

42. The method of claims 39, wherein the weight loss drug is Contrave (Naltrexone Hydrochloride and Bupropion Hydrochloride), Qysmia (Phentermine and Topiramate), or Belviq (lorcaserin hydrochloride).


SEQUENCE LISTING (15 sequences)

SEQ ID NO: 1 - protein, 11 aa, Artificial sequence
  Synthetic
YVMGHFRWDFG

SEQ ID NO: 2 - protein, 8 aa, Artificial sequence
  Synthetic
  MOD_RES (1)..(1): Xaa=Nle
XDPPARWK

SEQ ID NO: 3 - protein, 8 aa, Artificial sequence
  Synthetic
  MOD_RES (1)..(1): Xaa=Nle
XDWPARWK

SEQ ID NO: 4 - skipped

SEQ ID NO: 5 - protein, 4 aa, Artificial sequence
  Synthetic
HFRW

SEQ ID NO: 6 - protein, 13 aa, Artificial sequence
  Synthetic
SYSMEHFRWGKPV

SEQ ID NO: 7 - protein, 22 aa, Artificial sequence
  Synthetic
AEKKDEGPYRMEHFRWGSPPKD

SEQ ID NO: 8 - protein, 23 aa, Artificial sequence
  Synthetic
YVMGHFRWDRFDPRNSSAGGSAQ

SEQ ID NO: 9 - protein, 12 aa, Artificial sequence
  Synthetic
YVMGHFRWDRFG

SEQ ID NO: 10 - protein, 11 aa, Artificial sequence
  Synthetic
YVMGHFRWDRF

SEQ ID NO: 11 - protein, 38 aa, Artificial sequence
  Synthetic
SYSMEHFRWGKPVGKKRRPVKVYPNGAEDSAEAFPLEF

SEQ ID NO: 12 - protein, 4 aa, Artificial sequence
  Synthetic
  MISC_FEATURE (1)..(1): Xaa = any of the Xaa1 Tetrapeptide MC3R agonists in Table 3
  MISC_FEATURE (4)..(4): Xaa = any of the Xaa4 Tetrapeptide MC3R agonists in Table 3
XRFX

SEQ ID NO: 13 - protein, 3 aa, Artificial sequence
  Synthetic
RRF

SEQ ID NO: 14 - protein, 3 aa, Artificial sequence
  Synthetic
HRF

SEQ ID NO: 15 - protein, 4 aa, Artificial sequence
  Synthetic
RRFA